(19) United States
(12) Patent Application Publication
(10) Pub. No.: US 2023/0407328 A1
(43) Pub. Date: Dec. 21, 2023

(54) PROCESS FOR ENRICHING ADENO-ASSOCIATED VIRUS

(71) Applicant: BioMarin Pharmaceutical Inc., Novato, CA (US)

(72) Inventors: John MAGA, Novato, CA (US); Harmit VORA, Novato, CA (US); Rahul SHETH, Novato, CA (US); Daniel GOLD, Novato, CA (US); Anant RISHI, Newark, CA (US); Yanhong ZHANG, Novato, CA (US); Kieu TRAN, Novato, CA (US)

(73) Assignee: BioMarin Pharmaceutical Inc., Novato, CA (US)

(21) Appl. No.: 18/034,766

(22) PCT Filed: Nov. 2, 2021
(86) PCT No.: PCT/US2021/057716

§ 371 Date: May 1, 2023

Related U.S. Application Data
(60) Provisional application No. 63/108,629, filed on Nov. 2, 2020.

(51) Int. Cl. C12N 15/86; C07K 1/36

(52) U.S. Cl. CPC C12N 15/86; C07K 1/36; C12N 2750/14143; C12N 2750/14151

(57) ABSTRACT

The present invention provides process for enriching adeno-associated virus particles using anion exchange chromatography and zonal ultracentrifugation.

### FIELD OF THE INVENTION

The present invention is directed to a process for enriching adeno-associated virus (AAV) particles using anion exchange chromatography and zonal ultracentrifugation.

### BACKGROUND OF THE INVENTION

Adeno-associated viruses (AAV) are small, non-pathogenic satellite viruses that are believed to require a helper adenovirus for replication. AAVs are similar in structure to adenoviruses but have a smaller icosahedral nucleocapsid. AAV are non-enveloped viruses with single-stranded DNA genome with at least one inverted terminal repeat (ITR) at the termini. For example, the AAV2 serotype can have a single-stranded DNA genome of approximately 4.7-kilobases (kb), with two 145 nucleotide-long ITRs at the termini. The virus does not encode a polymerase and therefore relies on cellular polymerases for genome replication. The ITRs flank the two viral genes—rep (replication) and cap (capsid), encoding non-structural and structural proteins, respectively. The rep gene, through the use of two promoters and alternative splicing, encodes four regulatory proteins that are dubbed Rep78, Rep68, Rep52 and Rep40. These proteins are involved in AAV genome replication and packaging. The cap gene, through alternative splicing and initiation of translation, gives rise to three capsid proteins, VP1 (virion protein 1), VP2 and VP3. The molecular weight of VP1, VP2, and VP3 for AAV2 is 87, 72 and 62 kDa, respectively. These capsid proteins assemble into a near-spherical protein shell of 60 subunits. The AAV structural simplicity and non-pathogenic nature make recombinant AAV (rAAV) a useful gene therapy vector. AAV gene therapy vectors can infect both replicating and non-replicating cells and introduce transgenes without integrating into the genome of the host cell. rAAV vectors are often preferred due to their high titer, ability to infect a broad range of cells, mild immune response, and overall safety. rAAV gene therapy vectors have been found to be highly useful for a number of diseases including diabetes and other pancreatic disorders.

The production of rAAV particles for gene therapy also produces various impurities. Accordingly, there remains a need for processes that effectively purify the rAAV particles from the impurities.

### SUMMARY OF THE INVENTION

The present invention solves one or more problems of the prior art by providing, in at least one embodiment, a method for purifying rAAV particles for gene therapy or therapeutically effective rAAV particles is disclosed. Prior to purification, the therapeutically effective rAAV particles are in a composition that also includes AAV production impurities. The AAV production impurities include a first portion having a net charge different from the therapeutically effective rAAV particles and a second portion having a density different from the therapeutically effective rAAV particles. The method of at least one embodiment includes the steps of removing the first portion from the composition by anion-exchange chromatography (AEX) and removing the second portion from the composition by zonal ultracentrifugation (ZUC). After the AEX and ZUC steps, the composition is substantially devoid of AAV production impurities. In refinements, the first portion or second portion of AAV production impurities are therapeutically ineffective rAAV particles.

In at least one embodiment, a method for purifying rAAV particles for gene therapy or therapeutically effective rAAV particles is disclosed. Prior to purification, the therapeutically effective rAAV particles are in a composition that also includes therapeutically ineffective rAAV particles. The method of at least one embodiment includes removing at least some of the therapeutically ineffective rAAV particles from the composition by AEX. After the removal step, the method of at least one embodiment further includes processing the composition by ZUC. After the AEX and ZUC steps, the composition is substantially devoid of therapeutically ineffective rAAV particles.

### BRIEF DESCRIPTION OF THE DRAWINGS

FIGS. 1A and 1B show a particle distribution profile from an rAAV preparation separated by analytical ultracentrifugation.

FIGS. 2A, 2B, 3A, and 3B show the impact of an rAAV preparation containing light and heavy capsids on transgene expression in cells infected with the rAAV.

FIGS. 4, 5A, 5B, 6A, 6B, 7, 8A, 8B, 9A, 9B, 10, 11A, 11B, 11C, 11D, 12A, and 12B are images showing labelled light and heavy capsids infecting a HepG2 cell.

FIG. 13 is a flowchart of the steps of the purification methods of various embodiments.

FIG. 14 is a flowchart showing the steps of AEX processing of various embodiments.

FIG. 15 is a flowchart showing the steps of tangential flow filtration processing after AEX processing of various embodiments.

FIG. 16 is a flowchart showing the steps of ZUC processing of various embodiments.

FIG. 17 is a flowchart showing the steps of tangential flow filtration processing after AEX processing of various embodiments.

FIG. 18 is a Zeta potential analysis showing the difference in net charge between heavy capsids, ZUC light capsids, and AEX light capsids relative to pH.

FIG. 19 shows a particle distribution profile from an rAAV preparation after anion exchange chromatography. The rAAV preparation was separated by analytical ultracentrifugation.

FIG. 20 is a cryogenic electron microscopy image of light and heavy capsids from an rAAV preparation after anion exchange chromatography. The arrows indicate dense particles (i.e., heavy capsids) and “not dense” particles (i.e., light capsids).

FIG. 21 is a graph showing vector genome titers and densities of the different fractions of an rAAV preparation undergoing zonal ultracentrifugation.

FIG. 22 shows a particle distribution profile from an rAAV preparation after zonal ultracentrifugation. The rAAV preparation was separated by analytical ultracentrifugation.

FIGS. 23A and 23B are gels containing fractions of an rAAV preparation separated by zonal ultracentrifugation. FIG. 23A is a gel western blot stained for VP capsid proteins. FIG. 23B is an alkaline agarose gel containing DNA isolated from ultracentrifugation fractions.

FIG. 24 is a cryogenic electron microscopy image of light and heavy capsids from an rAAV preparation after zonal ultracentrifugation. The arrows indicate dense particles (i.e., heavy capsids) and “not dense” particles (i.e., light capsids).

FIGS. 25A and 25B show analysis of an rAAV preparation undergoing anion exchange chromatography and zonal ultracentrifugation. FIG. 25A shows the absorption spectrum of an rAAV preparation during anion exchange chromatography. FIG. 25B shows capsid and vector genome titers for the different fractions of the rAAV preparation during zonal ultracentrifugation.

FIG. 26 shows capsid titers for the different fractions of the rAAV preparation undergoing zonal ultracentrifugation with and without prior anion exchange chromatography processing.

FIGS. 27A, 27B, and 27C show an analysis of light capsids when subjected to anion exchange chromatography and zonal ultracentrifugation. FIG. 27A shows the absorption spectrum of an rAAV preparation during anion exchange chromatography. The circled peak in FIG. 27A was subsequently processed by zonal ultracentrifugation. FIG. 27B shows capsid and vector genome titers for the different fractions of the circled peak in FIG. 27A during zonal ultracentrifugation. The circled peak in FIG. 27B was again processed by anion exchange chromatography. FIG. 27C shows the absorption spectrum of the circled peak in FIG. 27B during anion exchange chromatography.

FIG. 28 shows a concentration of rAAV associated with Rep protein(s), which is an impurity, after immunochromatography purification using an affinity resin such as AVB Sepharose, after anion exchange chromatography processing, and after zonal ultracentrifugation processing. FIG. 28 highlights that anion exchange chromatography processing removed a substantial concentration of rAAV associated with Rep proteins from an AVB Sepharose purified composition comprising therapeutically effective rAAV. FIG. 28 further highlights that zonal ultracentrifugation processing further removed rAAV associated with Rep proteins that were not removed by anion exchange chromatography processing.

FIG. 29 shows the removal of rAAV associated with Rep protein(s) during anion exchange chromatography processing. After the composition has been processed by anion exchange chromatography, the concentration of Rep protein was assessed. Neither the eluate nor the wash contained substantial concentrations of Rep protein. Substantial concentrations of Rep protein were identified when the anion exchange chromatography column was regenerated to remove the impurities that remained after the load, wash, and elution steps.

FIG. 30 shows the removal of rAAV associated with Rep protein(s) during zonal ultracentrifugation processing. The isolated fractions (i.e., “Pool”) have significantly lower concentrations of Rep protein as compared to the fractions that were not isolated (i.e., “Post-pool”). It is also noted that concentrations of encapsulated vector genome are substantially increased in the pool fractions as compared to the post pool fractions.

FIG. 31 shows the removal of deamidated capsids, which is an impurity, after immunochromatography purification using an affinity resin such as AVB Sepharose, after anion exchange chromatography processing, and after zonal ultracentrifugation processing. FIG. 31 highlights that anion exchange chromatography processing removed a substantial concentration of deamidated capsids from an AVB Sepharose purified composition comprising therapeutically effective rAAV. FIG. 31 further highlights that zonal ultracentrifugation processing further removed deamidated capsids that were not removed by anion exchange chromatography processing.

FIG. 32 shows the removal of deamidated capsids during anion exchange chromatography processing. After the composition has been processed by anion exchange chromatography, the concentration of deamidated capsids were assessed. The eluate contained a substantially reduced concentration of deamidated capsids. Substantial concentrations of deamidated capsids were identified in the eluted wash buffer and when the anion exchange chromatography column was regenerated to remove the impurities that remained after the load, wash, and elution steps.

FIG. 33 shows the removal of deamidated capsids during zonal ultracentrifugation processing. The isolated fractions (i.e., “Pool”) have significantly lower concentrations of deamidated capsids as compared to the fractions that were not isolated (i.e., “Post-pool”). It is also noted that concentrations of encapsulated vector genome is substantially increased in the pool fractions as compared to the post pool fractions.

### DETAILED DESCRIPTION OF THE INVENTION

As required, detailed embodiments of the present disclosure are disclosed herein; however, it is to be understood that the disclosed embodiments are merely exemplary and may be embodied in various and alternative forms.

Except in the examples, or where otherwise expressly indicated, all numerical quantities in this description indicating amounts of material or conditions of reaction and/or use are to be understood as modified by the word “about”. For example, description referring to “about X” includes description of “X.” In one example, the term “about” is understood as within a range of normal tolerance in the art, for example within 2 standard deviations of the mean. In different examples, “about” refers a variability of ±0.0001%, ±0.0005%, ±0.001%, ±0.005%, ±0.01%, ±0.05%, ±0.1%, ±0.5%, ±1%, ±5%, or ±10%. In further examples, “about” can be understood as within ±9%, ±8%, ±7%, ±6%, ±5%, ±4%, ±3%, or ±2%.

Unless otherwise clear from context, all numerical values provided herein are modified by the term about. All ranges include the endpoints of the ranges. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “with”, “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

As used herein, the terms “heterologous gene”, “heterologous sequence”, “heterologous”, “heterologous regulatory sequence”, “heterologous transgene”, or “transgene” means that the referenced gene or regulatory sequence is not naturally present in the AAV vector or particle and has been artificially introduced therein. For example, these terms refer to a nucleic acid that comprises both a heterologous gene and a heterologous regulatory sequence that are operably linked to the heterologous gene that control expression of that gene in a host cell. It is contemplated that the transgene herein can encode a biomolecule (e.g., a therapeutic biomolecule), such as a protein (e.g., an enzyme), polypeptide, peptide, RNA (e.g., tRNA, dsRNA, ribosomal RNA, catalytic RNAs, siRNA, miRNA, pre-miRNA, lncRNA, snoRNA, small hairpin RNA, trans-splicing RNA, and antisense RNA), one or more components of a gene or base editing system, e.g., a CRISPR gene editing system, antisense oligonucleotides (AONs), antisense oligonucleotide (AON)-mediated exon skipping, a poison exon(s) that triggers nonsense mediated decay (NMD), or a dominant negative mutant.

The term “vector” is understood to refer to any genetic element, such as a nucleic acid molecule, plasmid, phage, transposon, cosmid, bacmid, mini-plasmid (e.g., plasmid devoid of bacterial elements), Doggybone DNA (e.g., minimal, closed-linear constructs), chromosome, virus, virion, etc., which is capable of replication when associated with the proper control elements and which can transfer gene sequences between cells. “Expression vector” refers to a vector including a recombinant polynucleotide including expression control sequences operatively linked to a nucleotide sequence to be expressed. An expression vector includes sufficient cis-acting elements for expression; other elements for expression can be supplied by the host cell or in vitro expression system. Expression vectors include all those known in the art, such as cosmids, plasmids (e.g., naked or contained in liposomes), artificial chromosomes, and viruses that incorporate the recombinant polynucleotide.

The term “recombinant” refers nucleic acid molecules or proteins formed by using recombinant DNA techniques. For example, a recombinant nucleic acid molecule can be formed by combining nucleic acid sequences and sequence elements. A recombinant protein can be a protein that is produced by a cell that has received a recombinant nucleic acid molecule.

The terms “encodes,” “encoded” and “encoding” refer to the inherent property of specific sequences of nucleotides in a nucleic acid molecule, such as a gene, complementary DNA (cDNA), or messenger RNA (mRNA), to serve as templates for synthesis of other polymers and macromolecules in biological processes. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA.

The present invention is directed to a method of purifying rAAV particles for gene therapy or therapeutically effective rAAV particles. The therapeutically effective rAAV particles include rAAV particles disclosed in or may be made according to knowing methods, e.g., as disclosed in U.S. Pat. No. 9,504,762, WO 2019/222136, US 2019/0376081, and WO 2019/217513, the disclosures of which are hereby incorporated in their entirety by reference.

In accordance with the present invention, the production of rAAV particles is an inefficient process that produces various impurities including therapeutically ineffective rAAV particles. These impurities limit the ability of purification techniques to further separate therapeutically effective rAAV particles from the impurities. To this end, the inventors have solved the limitations in the current state of the art by developed methods of isolating therapeutically effective rAAV particles from the impurities.

In various embodiments, methods and processes of purifying therapeutically effective rAAV particles from a composition including therapeutically effective rAAV particles and AAV production impurities including therapeutically ineffective rAAV particles. The composition of various embodiments is a production of rAAV particles. The AAV production impurities can also include impurities having a net charge different from the therapeutically effective rAAV particles or impurities having a density different from the AAV particles.

The methods and processes of various embodiments include subjecting the composition to AEX, where the impurities having a net charge different from the therapeutically effective rAAV particles are removed from the composition. These impurities include therapeutically ineffective rAAV particles. It was discovered that the use of ZUC for purification of therapeutically effective rAAV particles is substantially limited due to the presence of therapeutically ineffective rAAV particles that are less soluble than therapeutically effective rAAV particles and prone to aggregation. Although not wishing to be bound by theory, these properties can overload the capacity of ZUC to isolate therapeutically effective rAAV particles. For example, the therapeutically ineffective rAAV particles without AEX processing may precipitate during ZUC and prevent separation from therapeutically effective rAAV particles. To this end, AEX removes a sufficient quantity of therapeutically ineffective rAAV particles from the composition such that a composition having increased concentrations of therapeutically effective rAAV particles can be efficiently loaded and processed by ZUC. For example, the increased concentrations of therapeutically effective rAAV particles are an economically viable quantity that can be processed each time by ZUC. For example, AEX processing can allow titers of at least 0.1×10e16 vector genome (vg) per load to 10×10e17 vg per load to be processed by ZUC. In various embodiments, the therapeutically ineffective rAAV particles includes capsids having associated Rep proteins. In other embodiments, the therapeutically ineffective rAAV particles includes capsids with one or more VP1 proteins having a deamidated N-terminal amino acid.

In various refinements, AEX removes or removes at least 85%, 86%, 87%, 88%, 89%, 90%, 91%, 92%, 93%, 94%, 95%, 96%, 97%, 98%, 99%, 99+%, or 100% of the impurities having a net charge different from the therapeutically effective rAAV particles. In other refinements, the percentage of impurities removed by AEX is a range between any two percentages provided above. In various refinements, AEX removes or removes at least 0.1%, 0.5%, 1%, 5%, 10%, 20%, 30%, 40%, 50%, 60%, 70%, 80%, 90%, 95%, 99%, or 99%+of therapeutically ineffective rAAV particles from the composition. In other refinements, the percentage of therapeutically ineffective rAAV particles removed from the composition by AEX is a range between any two percentages provided above.

In various refinements, AEX allows for subsequent processing of the composition by ZUC or allows the original composition to have a greater quantity of therapeutically effective rAAV particles that are processed by ZUC.

In various refinements, AEX reduces a contaminating virus concentration in the composition by at least a Logio value of at least 2, at least 2.1, at least 2.2, at least 2.3, at least 2.4, at least 2.5, at least 2.6, at least 2.7, at least 2.8, at least 2.9, at least 3, at least 3.1, at least at least 3.2, at least 3.3, at least 3.4, at least 3.5, at least 3.6, at least 3.7, at least 3.8, at least 3.9, at least 4, at least 4.1, at least 4.2, at least 4.3, at least 4.4, at least 4.5, at least 4.6, at least 4.7, at least 4.8, at least 4.9, at least 5, at least 5.1, at least 5.2, at least 5.3, at least 5.4, at least at least 5.6, at least 5.7, at least 5.8, at least 5.9, at least 6, at least 6.1, at least 6.2, at least 6.3, at least 6.4, at least 6.5, at least 6.6, at least 6.7, at least 6.8, at least 6.9, or at least 7.

In various refinements, the AEX step includes processing the composition through a membrane filter or column containing a strong basic anion exchange resin(s). Examples of strong basic anion exchange resins include quaternized polyethyleneimine, Type I resins have trimethyl ammonium groups such as trimethyl-ammoniumethyl (TMAE), and Type II resins have dimethylethanolamine groups such as diethyl aminoethyl (DEAE). Examples of filters or columns that have strong basic anion exchange resins include Mustang Q (Pall), Sartobind Q (Sartorius), POROS 50 HQ (Thermofisher), POROS 50 XQ (Thermofisher), Fractogel TMAE (EMD Millipore), Fractogel DEAE (EMD Millipore), Eshmuno Q (EMD Millipore), CIMmultus-QA (BIA separations), Nuvia Q (Bio-Rad), Q Sepharose XL (Cytiva), Q Sepharose HP (Cytiva), Capto Q Impres (Cytiva), Source 15Q (Cytiva), Source 30Q (Cytiva), Mono Q (Cytiva), TSKgel Q-STAT (TOSOH bioscience), TSKgel SuperQ-5PW (20) (Tosoh Biosciences), Toyopearl SuperQ 650M (Tosoh Biosciences), Toyopearl GigaCap Q 650M (Tosoh Biosciences), and Capto Adhere Impres (Multimodal, Cytiva). Examples of weak basic anion exchange resins include Diethylaminoethyl (DEAE), Dimethylaminopropyl, or Diethylaminopropyl (ANX). Examples of filters and columns that have weak basic anion exchange resins include Sartobind STIC PA (Sartorius), DEAE Sepharose FF (Cytiva), Poros 50 D (Thermofisher), POROS 50PI (ThermoFisher), Fractogel EMD DEAE (M) (EMD Millipore), MacroPrep DEAE Support (Bio-Rad), DEAE Ceramic HyperD 20 (Sartorius), Toyopearl NH2-750F (Tosoh Biosciences), or Toyopearl DEAE 650 M (Sigma Aldrich). Suitable buffers and buffering agents for use with AEX may include ions contributed from a variety of sources, such as, e.g., N-methylpiperazine; piperazine, Bis-tris(hydroxymethyl)aminomethane (Tris), Bis-Tris propane, MES, Hepes, BTP; an or a phosphate buffer N-methyldiethanolamine; 1,3-diaminopropane; ethanolamine; acetic acid such as sodium acetate or lithium acetate; or citrates and the like. In various refinements, the bed height of the column is at least 7 centimeters (cm), 7 cm, 7.1 cm, 7.2 cm, 7.3 cm, 7.4 cm, 7.5 cm, 7.6 cm, 7.7 cm, 7.8 cm, 7.9 cm, 8 cm, 8.1 cm, 8.2 cm, 8.3 cm, 8.4 cm, 8.5 cm, 8.6 cm, 8.7 cm, 8.8 cm, 8.9 cm, 9 cm, 9.1 cm, 9.2 cm, 9.3 cm, 9.4 cm, 9.5 cm, 9.6 cm, 9.7 cm, 9.8 cm, 9.9 cm, 10 cm, 10.1 cm, 10.2 cm, 10.3 cm, 10.4 cm, 10.5 cm, 10.6 cm, 10.7 cm, 10.8 cm, 10.9 cm, 11 cm, 11.1 cm, 11.2 cm, 11.3 cm, 11.4 cm, 11.5 cm, 11.6 cm, 11.7 cm, 11.8 cm, 11.9 cm, 12 cm, 12.1 cm, 12.2 cm, 12.3 cm, 12.4 cm, 12.5 cm, 12.6 cm, 12.7 cm, 12.8 cm, 12.9 cm, 13 cm, 13.1 cm, 13.2 cm, 13.3 cm, 13.4 cm, 13.5 cm, 13.6 cm, 13.7 cm, 13.8 cm, 13.9 cm, 14 cm, 14.1 cm, 14.2 cm, 14.3 cm, 14.4 cm, 14.5 cm, 14.6 cm, 14.7 cm, 14.8 cm, 14.9 cm, or 15 cm. In other refinements, the bed height of the column is greater than 15 cm (15.1 cm, 15.2 cm, 15.3 cm, 15.4 cm, 15.5 cm, 15.6 cm, 15.7 cm, 15.8 cm, 15.9 cm, 16 cm, 16.1 cm, 16.2 cm, 16.3 cm, 16.4 cm, 16.5 cm, 16.6 cm, 16.7 cm, 16.8 cm, 16.9 cm, 17 cm, 17.1 cm, 17.2 cm, 17.3 cm, 17.4 cm, 17.5 cm, 17.6 cm, 17.7 cm, 17.8 cm, 17.9 cm, 18 cm, 18.1 cm, 18.2 cm, 18.3 cm, 18.4 cm, 18.5 cm, 18.6 cm, 18.7 cm, 18.8 cm, 18.9 cm, 19 cm, 19.1 cm, 19.2 cm, 19.3 cm, 19.4 cm, 19.5 cm, 19.6 cm, 19.7 cm, 19.8 cm, 19.9 cm, 20 cm, 20.1 cm, 20.2 cm, 20.3 cm, 20.4 cm, 20.5 cm, 20.6 cm, 20.7 cm, 20.8 cm, 20.9 cm, 21 cm, 21.1 cm, 21.2 cm, 21.3 cm, 21.4 cm, 21.5 cm, 21.6 cm, 21.7 cm, 21.8 cm, 21.9 cm, 22 cm, 22.1 cm, 22.2 cm, 22.3 cm, 22.4 cm, 22.5 cm, 22.6 cm, 22.7 cm, 22.8 cm, 22.9 cm, 23 cm, 23.1 cm, 23.2 cm, 23.3 cm, 23.4 cm, 23.5 cm, 23.6 cm, 23.7 cm, 23.8 cm, 23.9 cm, 24 cm, 24.1 cm, 24.2 cm, 24.3 cm, 24.4 cm, 24.5 cm, 24.6 cm, 24.7 cm, 24.8 cm, 24.9 cm, 25 cm, 25.1 cm, 25.2 cm, 25.3 cm, 25.4 cm, 25.5 cm, 25.6 cm, 25.7 cm, 25.8 cm, 25.9 cm, 26 cm, 26.1 cm, 26.2 cm, 26.3 cm, 26.4 cm, 26.5 cm, 26.6 cm, 26.7 cm, 26.8 cm, 26.9 cm, 27 cm, 27.1 cm, 27.2 cm, 27.3 cm, 27.4 cm, 27.5 cm, 27.6 cm, 27.7 cm, 27.8 cm, 27.9 cm, 28 cm, 28.1 cm, 28.2 cm, 28.3 cm, 28.4 cm, 28.5 cm, 28.6 cm, 28.7 cm, 28.8 cm, 28.9 cm, 29 cm, 29.1 cm, 29.2 cm, 29.3 cm, 29.4 cm, 29.5 cm, 29.6 cm, 29.7 cm, 29.8 cm, 29.9 cm, 30 cm). In other refinements, the bed height of the column is range between any two bed heights provided above.

In various refinements, the AEX operation is conducted at a pH of at least 6, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11. In other refinements, the pH at which the AEX operation is conducted is a range between any two pH provided above.

In various refinements, the AEX operation is conducted at a temperature of at least 4 degrees Celsius (° C.), 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., or 36° C. In other refinements, the temperature at which the AEX operation is conducted is a range between any two temperatures provided above.

In various refinements, the composition loaded onto a AEX column for AEX processing has a titer of at least 0.1×10e16 vector genome per liter (vg/L), 015×10e16 vg/L, 0.5×10e16 vg/L, 1×10e16 vg/L, 1.5×10e16 vg/L, 2×10e16 vg/L, 2.5×10e16 vg/L, 3×10e16 vg/L, 3.5×10e16 vg/L, 4×10e16 vg/L, 4.5×10e16 vg/L, 5×10e16 vg/L, 5.5×10e16 vg/L, 6×10e16 vg/L, 6.5×10e16 vg/L, 7×10e16 vg/L, 7.5×10e16 vg/L, 8×10e16 vg/L, 8.5×10e16 vg/L, 9×10e16 vg/L, 9.5×10e16 vg/L, or 10×10e16 vg/L. In other refinements, the titer is a range between any two titers provided above.

In various refinements, the composition loaded onto an AEX column for AEX processing has a conductivity of at least 0.0 millisiemens/centimeters (mS/cm), 0.0 mS/cm, 0.001 mS/cm, 0.002 mS/cm, 0.003 mS/cm, 0.004 mS/cm, 0.005 mS/cm, 0.006 mS/cm, 0.007 mS/cm, 0.008 mS/cm, 0.009 mS/cm, 0.01 mS/cm, 0.02 mS/cm, 0.03 mS/cm, 0.04 mS/cm, 0.05 mS/cm, 0.06 mS/cm, 0.07 mS/cm, 0.08 mS/cm, 0.09 mS/cm, 0.1 mS/cm, 0.1 mS/cm, 0.2 mS/cm, 0.3 mS/cm, 0.4 mS/cm, 0.5 mS/cm, 0.6 mS/cm, 0.7 mS/cm, 0.8 mS/cm, 0.9 mS/cm, 1 mS/cm, 1.1 mS/cm, 1.2 mS/cm, 1.3 mS/cm, 1.4 mS/cm, 1.5 mS/cm, 1.6 mS/cm, 1.7 mS/cm, 1.8 mS/cm, 1.9 mS/cm, 2 mS/cm, 2.1 mS/cm, 2.2 mS/cm, 2.3 mS/cm, 2.4 mS/cm, 2.5 mS/cm, 2.6 mS/cm, 2.7 mS/cm, 2.8 mS/cm, 2.9 mS/cm, 3 mS/cm, 3.1 mS/cm, 3.2 mS/cm, 3.3 mS/cm, 3.4 mS/cm, 3.5 mS/cm, 3.6 mS/cm, 3.7 mS/cm, 3.8 mS/cm, 3.9 mS/cm, 4 mS/cm, 4.1 mS/cm, 4.2 mS/cm, 4.3 mS/cm, 4.4 mS/cm, 4.5 mS/cm, 4.6 mS/cm, 4.7 mS/cm, 4.8 mS/cm, 4.9 mS/cm, 5 mS/cm, 5.1 mS/cm, 5.2 mS/cm, 5.3 mS/cm, 5.4 mS/cm, 5.5 mS/cm, 5.6 mS/cm, 5.7 mS/cm, 5.8 mS/cm, 5.9 mS/cm, 6 mS/cm, 6.1 mS/cm, 6.2 mS/cm, 6.3 mS/cm, 6.4 mS/cm, 6.5 mS/cm, 6.6 mS/cm, 6.7 mS/cm, 6.8 mS/cm, 6.9 mS/cm, or 7 mS/cm. In other refinements, the composition loaded onto an AEX column for AEX processing has a conductivity of less than 1 mS/cm. In other refinements, the conductivity of the composition loaded onto an AEX column is a range between any two conductivities provided above.

In various refinements, the composition loaded onto a AEX column for AEX processing has a pH of at least 7, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.5, 10.6, 10.7, 10.8, 10.9, or 11. In other refinements, the pH of the composition loaded onto the AEX column is a range between any two pH provided above.

In various refinements, the column is washed with a buffer after running the composition through the AEX column. In various refinements, the conductivity of the wash buffer is at least 1 mS/cm, 1 mS/cm, 1.1 mS/cm, 1.2 mS/cm, 1.3 mS/cm, 1.4 mS/cm, 1.5 mS/cm, 1.6 mS/cm, 1.7 mS/cm, 1.8 mS/cm, 1.9 mS/cm, 2 mS/cm, 2.1 mS/cm, 2.2 mS/cm, 2.3 mS/cm, 2.4 mS/cm, 2.5 mS/cm, 2.6 mS/cm, 2.7 mS/cm, 2.8 mS/cm, 2.9 mS/cm, 3 mS/cm, 3.1 mS/cm, 3.2 mS/cm, 3.3 mS/cm, 3.4 mS/cm, 3.5 mS/cm, 3.6 mS/cm, 3.7 mS/cm, 3.8 mS/cm, 3.9 mS/cm, 4 mS/cm, 4.1 mS/cm, 4.2 mS/cm, 4.3 mS/cm, 4.4 mS/cm, 4.5 mS/cm, 4.6 mS/cm, 4.7 mS/cm, 4.8 mS/cm, 4.9 mS/cm, 5 mS/cm, 5.1 mS/cm, 5.2 mS/cm, 5.3 mS/cm, 5.4 mS/cm, 5.5 mS/cm, 5.6 mS/cm, 5.7 mS/cm, 5.8 mS/cm, 5.9 mS/cm, 6 mS/cm, 6.1 mS/cm, 6.2 mS/cm, 6.3 mS/cm, 6.4 mS/cm, 6.5 mS/cm, 6.6 mS/cm, 6.7 mS/cm, 6.8 mS/cm, 6.9 mS/cm, or 7 mS/cm. In other refinements, the conductivity of the wash buffer is greater than 7 mS/cm. In other refinements, the conductivity of the wash buffer is a range between any two conductivities provided above.

As the column is washed with the wash buffer of various embodiments, monitoring the ultraviolet (UV) absorbances at the 260 nanometer (nm) and 280 nm wavelength of the wash buffer exiting the column and calculating the ratio of the 260 nm wavelength to 280 nm wavelength (A₂₆₀:A₂₈₀) can eliminate human error and variation between different purifications. In various refinements, the A₂₆₀:A₂₈₀ of the wash buffer exiting the column is at least 0.5, 0.5, 0.6, 0.7, 0.8, 0.9, 1, 1.1, 1.2, 1.3, 1.4, or 1.5. In other refinements, the A260:A28o ratio of the wash buffer exiting the column is a range between any two ratios provided above.

In various refinements, the composition is eluted with an elution buffer containing a concentration of a buffering agent after washing the AEX column with the wash buffer. In various refinements, the concentration of the buffering agent is at least 0.5 mM, 0.5 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 134 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, or 175 mM. In other refinements, the concentration of the buffering agent is a range between any two concentration provided above. The elution buffer of various refinements also has a conductivity of at least 1 mS/cm, 1 mS/cm, 1.1 mS/cm, 1.2 mS/cm, 1.3 mS/cm, 1.4 mS/cm, 1.5 mS/cm, 1.6 mS/cm, 1.7 mS/cm, 1.8 mS/cm, 1.9 mS/cm, 2 mS/cm, 2.1 mS/cm, 2.2 mS/cm, 2.3 mS/cm, 2.4 mS/cm, 2.5 mS/cm, 2.6 mS/cm, 2.7 mS/cm, 2.8 mS/cm, 2.9 mS/cm, 3 mS/cm, 3.1 mS/cm, 3.2 mS/cm, 3.3 mS/cm, 3.4 mS/cm, 3.5 mS/cm, 3.6 mS/cm, 3.7 mS/cm, 3.8 mS/cm, 3.9 mS/cm, 4 mS/cm, 4.1 mS/cm, 4.2 mS/cm, 4.3 mS/cm, 4.4 mS/cm, 4.5 mS/cm, 4.6 mS/cm, 4.7 mS/cm, 4.8 mS/cm, 4.9 mS/cm, 5 mS/cm, 5.1 mS/cm, 5.2 mS/cm, 5.3 mS/cm, 5.4 mS/cm, 5.5 mS/cm, 5.6 mS/cm, 5.7 mS/cm, 5.8 mS/cm, 5.9 mS/cm, 6 mS/cm, 6.1 mS/cm, 6.2 mS/cm, 6.3 mS/cm, 6.4 mS/cm, 6.5 mS/cm, 6.6 mS/cm, 6.7 mS/cm, 6.8 mS/cm, 6.9 mS/cm, 7 mS/cm, 7.1 mS/cm, 7.2 mS/cm, 7.3 mS/cm, 7.4 mS/cm, 7.5 mS/cm, 7.6 mS/cm, 7.7 mS/cm, 7.8 mS/cm, 7.9 mS/cm, 8 mS/cm, 8.1 mS/cm, 8.2 mS/cm, 8.3 mS/cm, 8.4 mS/cm, 8.5 mS/cm, 8.6 mS/cm, 8.7 mS/cm, 8.8 mS/cm, 8.9 mS/cm, 9 mS/cm, 9.1 mS/cm, 9.2 mS/cm, 9.3 mS/cm, 9.4 mS/cm, 9.5 mS/cm, 9.6 mS/cm, 9.7 mS/cm, 9.8 mS/cm, 9.9 mS/cm, or 10 mS/cm. In other refinements, the conductivity of the elution buffer is a range between any two conductivities provided above.

In various refinements, AEX operation include processing of the composition through the AEX column, wash step, or elution step is conducted at a flow rate of at least 50 centimeter/hour (cm/hr), 50 cm/hr, 55 cm/hr, 60 cm/hr, 65 cm/hr, 70 cm/hr, 75 cm/hr, 80 cm/hr, 85 cm/hr, 90 cm/hr, 95 cm/hr, 100 cm/hr, 105 cm/hr, 110 cm/hr, 115 cm/hr, 120 cm/hr, 125 cm/hr, 130 cm/hr, 135 cm/hr, 140 cm/hr, 145 cm/hr, 150 cm/hr, 155 cm/hr, 160 cm/hr, 170 cm/hr, 180 cm/hr, 190 cm/hr, 200 cm/hr, 210 cm/hr, 220 cm/hr, 230 cm/hr, 240 cm/hr, 250 cm/hr, 260 cm/hr, 270 cm/hr, 280 cm/hr, 290 cm/hr, or 300 cm/hr. In other refinements, the flowrate at which the AEX operation is conducted is a range between any two flow rates provided above.

During the elution step, collection of the composition starts when the composition being eluted reaches a UV absorbance at the 260 nm wavelength (A26o) at an optical length. In various refinements, the collection of the composition starts when the composition being eluted reaches at least 0.1 absorbance units (AU)/cm, 0.1 AU/cm, 0.15 AU/cm, 0.2 AU/cm, 0.25 AU/cm, 0.3 AU/cm, 0.35 AU/cm, 0.4 AU/cm, 0.45 AU/cm, 0.5 AU/cm, 0.55 AU/cm, 0.6 AU/cm, 0.65 AU/cm, 0.7 AU/cm, 0.75 AU/cm, 0.8 AU/cm, 0.85 AU/cm, 0.9 AU/cm, 0.95 AU/cm, or 1 AU/cm. In other refinements, the absorbance when collection start is a range between any two absorbances provided above.

During the elution step, collection of the composition ends when the composition being eluted has reached a A₂₆₀ that is a percentage of the maximum A₂₆₀ that the composition reaches. In various refinements, the percentage is at least 0.1%, 0.1%, 0.2%, 0.4%, 0.5%, 0.6%, 0.7%, 0.8%, 0.9%, 1%, 2%, 3%, 4%, 5%, 6%, 7%, 8%, 9% , 10%, 11%, 12%, 13%, 14%, 15%, 16%, 17%, 18%, 19%, 20%, 21%, 22%, 23%, 24%, or 25%. In other refinements, the percentage of the maximum A26o is range between any two percentages provided above.

In various refinements, the pH of the composition eluted from the AEX column or eluate is or is adjusted to at least 6, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11. In other refinements, the pH of the composition eluted from the AEX column is a range between any two pH provided above.

The methods and processes of various embodiments include subjecting the composition to ZUC, where the impurities having a density different from the therapeutically effective rAAV particles are removed from the composition.

In various refinements, the ZUC removes or removes at least 85%, 86%, 87%, 88%, 89%, 90%, 91%, 92%, 93%, 94%, 95%, 96%, 97%, 98%, 99%, 99+%, or 100% of impurities having a density different from the therapeutically effective rAAV particles. In other refinements, the percentage of impurities removed by ZUC is a range between any two percentages provided above. In various refinements, ZUC reduces a contaminating virus concentration in the composition by at least a Logio value of at least 1.5, at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3, at least 3.1, at least 3.2, at least 3.3, at least 3.4, at least 3.5, at least 3.6, at least 3.7, at least 3.8, at least 3.9, or at least 4.

In various refinements of ZUC processing, a gradient compound is added to the composition and the composition is loaded between a cushion layer and an overlay layer in a rotor for zonal ultracentrifugation. After ZUC has been completed, a displacement solution is pumped into the rotor to force the cushion layer, composition, and overlay layer from the ZUC rotor. Alternatively, the cushion layer, composition, and overlay layer is pumped from the ZUC rotor without using a displacement solution. In both loading and unloading the ZUC rotor with the layers, the ZUC rotor may be spinning or stationary. In ZUC processing, the overlay layer is first pumped into a spinning or stationary ZUC rotor, followed by the composition with the gradient compound and cushion layer. The cushion layer, overlay layer, and displacement solution also contain a gradient compound. Examples of gradient forming compositions include cesium chloride (CsCl), iodixanol, or sucrose. The cushion layer prevents particles (e.g., therapeutically effective rAAV) from pelleting against the wall of the rotor and the overlay layer prevents particles from migrating out of the gradient formed by the gradient compound. In refinements, the concentration of the gradient compound in the cushion layer, composition, and overlay layer is different from each other. In other refinements, the concentration of the gradient compound in the cushion layer is greater than the composition. In further refinements, the concentration of the gradient in the compound is greater than the overlay layer.

In various refinements, the concentration of the gradient compound in the cushion layer, composition, overlay layer, or displacement solution is at least 15%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%. In other refinements, the concentration of the gradient compound in the cushion layer, composition, overlay layer, or displacement solution is a range of any two concentrations provided above.

In various refinements, the weight of the overlay layer pumped into the ZUC rotor is at least 117 grams(g), 117 g, 118 g, 119 g, 120 g, 121 g, 122 g, 123 g, 124 g, 125 g, 126 g, 127 g, 128 g, 129 g, 130 g, 131 g, 132 g, 133 g, 134 g, 135 g, 136 g, 137 g, 138 g, 139 g, 140 g, 141 g, 142 g, 143 g, 144 g, 145 g, 146 g, 147 g, 148 g, 149 g, 150 g, 151 g, 152 g, 153 g, 154 g, 155 g, 156 g, 157 g, 158 g, 159 g, 160 g, 161 g, 162 g, 163 g, 164 g, 165 g, 166 g, 167 g, 168 g, 169 g, 170 g, 171 g, 172 g, 173 g, 174 g, 175 g, 176 g, 177 g, 178 g, 179 g, 180 g, 181 g, 182 g, 183 g, 184 g, 185 g, 186 g, 187 g, 188 g, 189 g, 190 g, 191 g, 192 g, 193 g, 194 g, 195 g, 196 g, 197 g, 198 g, 199 g, 200 g, 201 g, 202 g, 203 g, 204 g, 205 g, 206 g, 207 g, 208 g, 209 g, 210 g, 211 g, 212 g, 213 g, 214 g, 215 g, 216 g, 217 g, 218 g, 219 g, 220 g, 221 g, 222 g, 223 g, 224 g, 225 g, 226 g, 227 g, 228 g, 229 g, 230 g, 231 g, 232 g, 233 g, 234 g, 235 g, 236 g, 237 g, 238 g, 239 g, 240 g, 241 g, 242 g, 243 g, 244 g, 245 g, 246 g, 247 g, 248 g, 249 g, 250 g, 251 g, 252 g, 253 g, 254 g, 255 g, 256 g, 257 g, 258 g, 259 g, 260 g, 261 g, 262 g, 263 g, 264 g, 265 g, 266 g, 267 g, 268 g, 269 g, 270 g, 271 g, 272 g, 273 g, 274 g, 275 g, 276 g, 277 g, 278 g, 279 g, 280 g, 281 g, 282 g, 283 g, 284 g, 285 g, 286 g, 287 g, 288 g, 289 g, 290 g, 291 g, 292 g, 293 g, 294 g, 295 g, 296 g, 297 g, 298 g, 299 g, 300 g, 301 g, 302 g, 303 g, 304 g, 305 g, 306 g, 307 g, 308 g, 309 g, 310 g, 311 g, 312 g, 313 g, 314 g, 315 g, 316 g, 317 g, 318 g, 319 g, 320 g, 321 g, 322 g, 323 g, 324 g, 325 g, 326 g, 327 g, 328 g, 329 g, 330 g, 331 g, 332 g, 333 g, 334 g, 335 g, 336 g, 337 g, 338 g, 339 g, 340 g, or 341 g. In alternative refinements, the weight of the overlay layer pumped into the ZUC rotor is at least 1100 g, 1110g, 1120g, 1130g, 1140g, 1150g, 1160g, 1170g, 1180g, 1190g, 1200g, 1210 g, 1220 g, 1230 g, 1240 g, 1250 g, 1260 g, 1270 g, 1280 g, 1290 g, or 1300 g. In other refinements, the weight of the overlay layer pumped into the ZUC rotor is a range between any two weights provided above.

In various refinements, the weight of the cushion layer pumped into the ZUC rotor is at least 534 g, 534 g, 535 g, 536 g, 537 g, 538 g, 539 g, 540 g, 541 g, 542 g, 543 g, 544 g, 545 g, 546 g, 547 g, 548 g, 549 g, 550 g, 551 g, 552 g, 553 g, 554 g, 555 g, 556 g, 557 g, 558 g, 559 g, 560 g, 561 g, 562 g, 563 g, 564 g, 565 g, 566 g, 567 g, 568 g, 569 g, 570 g, 571 g, 572 g, 573 g, 574 g, 575 g, 576 g, 577 g, 578 g, 579 g, 580 g, 581 g, 582 g, 583 g, 584 g, 585 g, 586 g, 587 g, 588 g, 589 g, 590 g, 591 g, 592 g, 593 g, 594 g, 595 g, 596 g, 597 g, 598 g, 599 g, 600 g, 601 g, 602 g, 603 g, 604 g, 605 g, 606 g, 607 g, 608 g, 609 g, 610 g, 611 g, 612 g, 613 g, 614 g, 615 g, 616 g, 617 g, 618 g, 619 g, 620 g, 621 g, 622 g, 623 g, 624 g, 625 g, 626 g, 627 g, 628 g, 629 g, 630 g, 631 g, 632 g, 633 g, 634 g, 635 g, 636 g, 637 g, 638 g, 639 g, 640 g, 641 g, 642 g, 643 g, 644 g, 645 g, 646 g, 647 g, 648 g, 649 g, 650 g, 651 g, 652 g, 653 g, 654 g, 655 g, 656 g, 657 g, 658 g, 659 g, 660 g, 661 g, 662 g, 663 g, 664 g, 665 g, 666 g, 667 g, or 668 g. In alternative refinements, the weight of the cushion layer pumped into the ZUC rotor is at least 3600 g, 3600 g, 3601 g, 3602 g, 3603 g, 3604 g, 3605 g, 3606 g, 3607 g, 3608 g, 3609 g, 3610 g, 3611 g, 3612 g, 3613 g, 3614 g, 3615 g, 3616 g, 3617 g, 3618 g, 3619 g, 3620 g, 3621 g, 3622 g, 3623 g, 3624 g, 3625 g, 3626 g, 3627 g, 3628 g, 3629 g, 3630 g, 3631 g, 3632 g, 3633 g, 3634 g, 3635 g, 3636 g, 3637 g, 3638 g, 3639 g, 3640 g, 3641 g, 3642 g, 3643 g, 3644 g, 3645 g, 3646 g, 3647 g, 3648 g, 3649 g, 3650 g, 3651 g, 3652 g, 3653 g, 3654 g, 3655 g, 3656 g, 3657 g, 3658 g, 3659 g, 3660 g, 3661 g, 3662 g, 3663 g, 3664 g, 3665 g, 3666 g, 3667 g, 3668 g, 3669 g, 3670 g, 3671 g, 3672 g, 3673 g, 3674 g, 3675 g, 3676 g, 3677 g, 3678 g, 3679 g, 3680 g, 3681 g, 3682 g, 3683 g, 3684 g, 3685 g, 3686 g, 3687 g, 3688 g, 3689 g, 3690 g, 3691 g, 3692 g, 3693 g, 3694 g, 3695 g, 3696 g, 3697 g, 3698 g, 3699 g, or 3700 g. In other refinements, the weight of the cushion layer pumped into the ZUC rotor is a range between any two weights provided above.

In various refinements, the composition loaded into a ZUC rotor for ZUC processing has a titer of at least 0.1×10e16 vg/load, 0.1×10e16 vg/load, 0.5×10e16 vg/load, 1×10e16 vg/load, 1.5×10e16 vg/load, 2×10e16 vg/load, 2.5×10e16 vg/load, 3×10e16 vg/load, 3.5×10e16 vg/load, 4×10e16 vg/load, 4.5×10e16 vg/load, 5×10e16 vg/load, 5.5×10e16 vg/load, 6×10e16 vg/load, 6.5×10e16 vg/load, 7×10e16 vg/load, 7.5×10e16 vg/load, 8×10e16 vg/load, 8.5×10e16 vg/load, 9×10e16 vg/load, 9.5×10e16 vg/load, 10×10e16 vg/load, 0.1×10e17 vg/load, 0.5×10e17 vg/load, 1×10e17 vg/load, 1.5×10e17 vg/load, 2×10e17 vg/load, 2.5×10e17 vg/load, 3×10e17 vg/load, 3.5×10e17 vg/load, 4×10e17 vg/load, 4.5×10e17 vg/load, 5×10e17 vg/load, 5.5×10e17 vg/load, 6×10e17 vg/load, 6.5×10e17 vg/load, 7×10e17 vg/load, 7.5×10e17 vg/load, 8×10e17 vg/load, 8.5×10e17 vg/load, 9×10e17 vg/load, 9.5×10e17 vg/load, or 10×10e17 vg/load. In other refinements, the titer is a range between any two titers provided above.

In various refinements, the density of the composition loaded into a ZUC rotor for ZUC processing is at least 1.347 grams per milliliters (g/mL), 1.3471 g/mL, 1.3472 g/mL, 1.3473 g/mL, 1.3474 g/mL, 1.3475 g/mL, 1.3476 g/mL, 1.3477 g/mL, 1.3478 g/mL, 1.3479 g/mL, 1.348 g/mL, 1.3481 g/mL, 1.3482 g/mL, 1.3483 g/mL, 1.3484 g/mL, 1.3485 g/mL, 1.3486 g/mL, 1.3487 g/mL, 1.3488 g/mL, 1.3489 g/mL, 1.349 g/mL, 1.3491 g/mL, 1.3492 g/mL, 1.3493 g/mL, 1.3494 g/mL, 1.3495 g/mL, 1.3496 g/mL, 1.3497 g/mL, 1.3498 g/mL, 1.3499 g/mL, 1.35 g/mL, 1.3501 g/mL, 1.3502 g/mL, 1.3503 g/mL, 1.3504 g/mL, 1.3505 g/mL, 1.3506 g/mL, 1.3507 g/mL, 1.3508 g/mL, 1.3509 g/mL, 1.351 g/mL, 1.3511 g/mL, 1.3512 g/mL, 1.3513 g/mL, 1.3514 g/mL, 1.3515 g/mL, 1.3516 g/mL, 1.3517 g/mL, 1.3518 g/mL, 1.3519 g/mL, 1.352 g/mL, 1.3521 g/mL, 1.3522 g/mL, 1.3523 g/mL, 1.3524 g/mL, 1.3525 g/mL, 1.3526 g/mL, 1.3527 g/mL, 1.3528 g/mL, 1.3529 g/mL, 1.353 g/mL, 1.3531 g/mL, 1.3532 g/mL, 1.3533 g/mL, 1.3534 g/mL, 1.3535 g/mL, 1.3536 g/mL, 1.3537 g/mL, 1.3538 g/mL, 1.3539 g/mL, 1.354 g/mL, 1.3541 g/mL, 1.3542 g/mL, 1.3543 g/mL, 1.3544 g/mL, 1.3545 g/mL, 1.3546 g/mL, 1.3547 g/mL, 1.3548 g/mL, 1.3549 g/mL, 1.355 g/mL, 1.3551 g/mL, 1.3552 g/mL, 1.3553 g/mL, 1.3554 g/mL, 1.3555 g/mL, 1.3556 g/mL, 1.3557 g/mL, 1.3558 g/mL, 1.3559 g/mL, 1.356 g/mL, 1.3561 g/mL, 1.3562 g/mL, 1.3563 g/mL, 1.3564 g/mL, 1.3565 g/mL, 1.3566 g/mL, 1.3567 g/mL, 1.3568 g/mL, 1.3569 g/mL, 1.357 g/mL, 1.3571 g/mL, 1.3572 g/mL, 1.3573 g/mL, 1.3574 g/mL, 1.3575 g/mL, 1.3576 g/mL, 1.3577 g/mL, 1.3578 g/mL, 1.3579 g/mL, 1.358 g/mL, 1.3581 g/mL, 1.3582 g/mL, 1.3583 g/mL, 1.3584 g/mL, 1.3585 g/mL, 1.3586 g/mL, 1.3587 g/mL, 1.3588 g/mL, 1.3589 g/mL, 1.359 g/mL, 1.3591 g/mL, 1.3592 g/mL, 1.3593 g/mL, 1.3594 g/mL, 1.3595 g/mL, 1.3596 g/mL, 1.3597 g/mL, 1.3598 g/mL, 1.3599 g/mL, 1.36 g/mL, 1.3601 g/mL, 1.3602 g/mL, 1.3603 g/mL, 1.3604 g/mL, 1.3605 g/mL, 1.3606 g/mL, 1.3607 g/mL, 1.3608 g/mL, 1.3609 g/mL, 1.361 g/mL, 1.3611 g/mL, 1.3612 g/mL, 1.3613 g/mL, 1.3614 g/mL, 1, 1.3675 g/mL, 1.3676 g/mL, 1.3677 g/mL, 1.3678 g/mL, 1.3679 g/mL, 1.368 g/mL, 1.3681 g/mL, 1.3682 g/mL, 1.3683 g/mL, 1.3684 g/mL, 1.3685 g/mL, 1.3686 g/mL, 1.3687 g/mL, 1.3688 g/mL, 1.3689 g/mL, 1.369 g/mL, 1.3691 g/mL, 1.3692 g/mL, 1.3693 g/mL, 1.3694 g/mL, 1.3695 g/mL, 1.3696 g/mL, 1.3697 g/mL, 1.3698 g/mL, 1.3699 g/mL, 1.37 g/mL, 1.3701 g/mL, 1.3702 g/mL, 1.3703 g/mL, 1.3704 g/mL, 1.3705 g/mL, 1.3706 g/mL, 1.3707 g/mL, 1.3708 g/mL, 1.3709 g/mL, or 1.371 g/mL. In other refinements, the density of the composition loaded into a ZUC rotor for ZUC processing is a range between any two densities provided above.

In various refinements, the cushion layer, composition, overlay layer, or displacement solution is loaded into the ZUC rotor at a flow rate of at least 20 milliliters per minutes (mL/min), 20 mL/min, 21 mL/min, 22 mL/min, 23 mL/min, 24 mL/min, 25 mL/min, 26 mL/min, 27 mL/min, 28 mL/min, 29 mL/min, 30 mL/min, 31 mL/min, 32 mL/min, 33 mL/min, 34 mL/min, 35 mL/min, 36 mL/min, 37 mL/min, 38 mL/min, 39 mL/min, 40 mL/min, 41 mL/min, 42 mL/min, 43 mL/min, 44 mL/min, 45 mL/min, 46 mL/min, 47 mL/min, 48 mL/min, 49 mL/min, 50 mL/min, 51 mL/min, 52 mL/min, 53 mL/min, 54 mL/min, 55 mL/min, 56 mL/min, 57 mL/min, 58 mL/min, 59 mL/min, 60 mL/min, 61 mL/min, 62 mL/min, 63 mL/min, 64 mL/min, 65 mL/min, 66 mL/min, 67 mL/min, 68 mL/min, 69 mL/min, or 70 mL/min. In an alternative refinement, the cushion layer, composition, overlay layer, or displacement solution is loaded into the ZUC rotor at a flow rate of 200 mL/min or greater. In other refinements, the flow rate in which the cushion layer, composition, overlay layer, or displacement solution is loaded into the ZUC rotor is a range between any two flow rates provided above.

After loading, the loaded ZUC rotor is centrifuged at speeds and for a time period to form gradients and separate particles by densities.

In various refinements, the ZUC rotor is centrifuged at least 10000 revolutions per minute (rpm), 10000 rpm, 11000 rpm, 12000 rpm, 13000 rpm, 14000 rpm, 15000 rpm, 16000 rpm, 17000 rpm, 18000 rpm, 19000 rpm, 20000 rpm, 21000 rpm, 22000 rpm, 23000 rpm, 24000 rpm, 25000 rpm, 26000 rpm, 27000 rpm, 28000 rpm, 29000 rpm, 30000 rpm, 31000 rpm, 32000 rpm, 33000 rpm, 34000 rpm, 35000 rpm, 36000 rpm, 37000 rpm, 38000 rpm, 39000 rpm, 40000 rpm, 41000 rpm, 42000 rpm, 43000 rpm, 44000 rpm, 45000 rpm, 46000 rpm, 47000 rpm, 48000 rpm, 49000 rpm, or 50000 rpm. In other refinements, the speed at which the ZUC rotor is centrifuged is a range of between any two speeds provided above.

In alternative refinements, the ZUC rotor is centrifuged at least 50000 g forces (G), 50000 G, 55000 G, 60000 G, 65000 G, 70000 G, 75000 G, 80000 G, 85000 G, 90000 G, 95000 G, 100000 G, 105000 G, 110000 G, 115000 G, 120000 G, or 125000 G. In other alternative refinements, the speed at which the ZUC rotor is centrifuged is a range of between any two speeds provided above.

In various refinements, the ZUC rotor is centrifuged for at least 13 hours (hr), 13 hr, 13.5 hr, 14 hr, 14.5 hr, 15 hr,15.5 hr, 16 hr, 16.5 hr, 17 hr, 17.5 hr, 18 hr, 18.5 hr, 19 hr, 19.5 hr, 20 hr, 20.5 hr, 21 hr, 21.5 hr, 22 hr, 22.5 hr, 23 hr, 23.5 hr, 24 hr, 24.5 hr, or 25 hr. In other refinements, the time period in which the loaded ZUC rotor is centrifuged is a range between any two times provided above.

In various refinements, the loaded ZUC rotor is centrifuged at a temperature of at least 10° C., 10° C., 10.5° C., 11° C., 11.5° C., 12° C., 12.5° C., 13° C., 13.5° C., 14° C., 14.5° C., 15° C., 15.5° C., 16° C., 16.5° C., 17° C., 17.5° C., 18° C., 18.5° C., 19° C., 19.5° C., 20° C., 20.5° C., 21° C., 21.5° C., 22° C., 22.5° C., 23° C., 23.5° C., 24° C., 24.5° C., 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., or 36° C. In other refinements, the temperature at which the loaded ZUC rotor is centrifuged is a range between any two temperatures proved above.

After the particles have been separated by ultracentrifugation, the loaded rotor can be stationary or centrifuged at a speed (e.g., 1000 rpm, 1500 rpm, 2000 rpm, 2500 rpm, 3000 rpm, 3500 rpm, 4000 rpm, 4500 rpm, or 5000) in order to recover the ZUC processed composition by pumped the displacement solution into the rotor to push the cushion layer, composition, and overlay layer to from the ZUC rotor. In various refinements, the loaded rotor is centrifuged for at least 0 minutes (min), at least 1 minutes, 10 min, 20 min, 30 min, min, 50 min, 60 min, 70 min, 80 min, 90 min, 100 min, 110 min, 120 min, 130 min, 140 min, 150 min, 160 min, 170 min, 180 min, 190 min, 200 min, 210 min, 220 min, 230 min, 240 min, 250 min, 260 min, 270 min, 280 min, 290 min, 300 min, 310 min, 320 min, 330 min, 340 min, 350 min, or 360 min. In other refinements, the time the rotor centrifuged for is a range between any two times provided above.

In various embodiments, AEX and ZUC remove or removes at least 85%, 86%, 87%, 88%, 89%, 90%, 91%, 92%, 93%, 94%, 95%, 96%, 97%, 98%, 99%, 99+%, or 100% of AAV production impurities. In different embodiments, the percentage of AAV production impurities removed by AEX and ZUC is a range between any two percentages listed above. In a refinement, the composition is substantially devoid of AAV production impurities after AEX and ZUC processing. As discussed above, when rAAV is produced there is a mixture of heavy capsids containing the full transgene of interest; partial capsids containing a portion of the transgene of interest; and light capsids. As highlighted below, empty and light capsids have no therapeutic efficacy and increase the exposure of a patient to heterologous proteins, nucleic acids etc., thereby increasing the likelihood of adverse immune reactions in the patient. Therefore, it is preferable that empty and light capsids should be removed from the AAV as much as possible. The combined use of AEX and ZUC is capable of obtaining heavy and partial capsids of a 99+% purity, where ZUC processing follows AEX. In this regard, if AEX is not used as a first step the empty and light capsids overload the capacity of the ZUC and result in precipitation during ZUC processing. On the other hand, if AEX is used without ZUC, empty and light capsids are not fully removed. Thus, the present invention is directed to methods of purifying AAV heavy and capsids, which are at least 85% pure (i.e., free from light and empty capsids). In further refinements, the heavy and partial capsids are at least 90% pure, the heavy and partial capsids are 99+% pure, or the composition has no detectable light or empty capsids.

The method and processes of various embodiments include processing the composition via tangential flow filtration (TFF) between AEX and ZUC processing. This TFF step includes the steps of ultrafiltration and diafiltration, where the AEX elution buffer is removed from the composition and replaced with a loading buffer including the gradient forming compound for ZUC processing.

In various refinements, the AEX processed composition loaded for TFF has a titer of at least 0.1×10e17 vg/squared meter (m 2), 0.1×10e17 vg/m², 0.5×10e17 vg/m², 1×10e17 vg/m², 1.5×10e17 vg/m², 2×10e17 vg/m², 2.5×10e17 vg/m², 3×10e17 vg/m², 3.5×10e17 vg/m², 4×10e17 vg/m², 4.5×10e17 vg/m², 5×10e17 vg/m², 5.5×10e17 vg/m², 6×10e17 vg/m², 6.5×10e17 vg/m², 7×10e17 vg/m², 7.5×10e17 vg/m², 8×10e17 vg/m², 8.5×10e17 vg/m², 9×10e17 vg/m², 9.5×10e17 vg/m 2 , or 10×10e17 vg/m 2 . In other refinements, the titer is a range between any two titers provided above.

In various refinements, the TFF filters the AEX processed composition at a transmembrane pressure (TMP) of at least 2 pounds per square inch (psi) (0.137895 bar), 2 psi (0.137895 bar), 3 psi (0.206843 bar), 4 psi (0.27579 bar), 5 psi (0.344738 bar), 6 psi (0.413685 bar), 7 psi (0.482633 bar), 8 psi (0.551581 bar), 9 psi (0.620528 bar), 10 psi (0.689476 bar), 11 psi (0.758423 bar), 12 psi (0.827371 bar), 13 psi (0.896318 bar), 14 psi (0.965266 bar), 15 psi (1.03421 bar), 16 psi (1.10316 bar), 17 psi (1.17211 bar), 18 psi (1.24106 bar), 19 psi (1.31 bar), 20 psi (1.37895 bar), 21 psi (1.4479 bar), 22 psi (1.51685 bar), 23 psi (1.58579 bar), 24 psi (1.65474 bar), 25 psi (1.72369 bar), 26 psi (1.79264 bar), 27 psi (1.86158 bar), 28 psi (1.93053 bar), 29 psi (1.99948 bar), 30 psi (2.06843 bar), 31 psi (2.13737 bar), 32 psi (2.20632 bar), 33 psi (2.27527 bar), 34 psi (2.34422 bar), 35 psi (2.41317 bar), 36 psi (2.48211 bar), 37 psi (2.55106 bar), 38 psi (2.62001 bar), 39 psi (2.68896 bar), 40 psi (2.7579 bar), 41 psi (2.82685 bar), 42 psi (2.8958 bar), 43 psi (2.96475 bar), 44 psi (3.03369 bar), 45 psi (3.10264 bar), 46 psi (3.17159 bar), 47 psi (3.24054 bar), 48 psi (3.30948 bar), 49 psi (3.37843 bar), or 50 psi (3.44738 bar). In other refinements, the TMP of the TFF for the AEX processed composition is range between any two TMPs provided above.

In various refinements, the TFF filters the AEX processed composition with a crossflow or retentate flow of at least 1 L/min/m², 1 L/min/m², 2 L/min/m², 3 L/min/m², 4 L/min/m², 5 L/min/m², 6 L/min/m², 7 L/min/m², 8 L/min/m², 9 L/min/m², 10 L/min/m², 11 L/min/m², 12 L/min/m², 13 L/min/m², 14 L/min/m 2 , or 15 L/min/m 2 . In other refinements, the crossflow of the TFF for the AEX processed composition is a range between any two crossflows provided above.

In various refinements, the TFF filters the AEX processed composition to a retentate concentration of at least 1×10e13 vg/mL, 1×10e13 vg/mL, 1.1×10e13 vg/mL, 1.2×10e13 vg/mL, 1.3×10e13 vg/mL, 1.4×10e13 vg/mL, 1.5×10e13 vg/mL, 1.6×10e13 vg/mL, 1.7×10e13 vg/mL, 1.8×10e13 vg/mL, 1.9×10e13 vg/mL, 2×10e13 vg/mL, 2.1×10e13 vg/mL, 2.2×10e13 vg/mL, 2.3×10e13 vg/mL, 2.4×10e13 vg/mL, 2.5×10e13 vg/mL, 2.6×10e13 vg/mL, 2.7×10e13 vg/mL, 2.8×10e13 vg/mL, 2.9×10e13 vg/mL, 3×10e13 vg/mL, 3.1×10e13 vg/mL, 3.2×10e13 vg/mL, 3.3×10e13 vg/mL, 3.4×10e13 vg/mL, 3.5×10e13 vg/mL, 3.6×10e13 vg/mL, 3.7×10e13 vg/mL, 3.8×10e13 vg/mL, 3.9×10e13 vg/mL, 4×10e13 vg/mL, 4.1×10e13 vg/mL, 4.2×10e13 vg/mL, 4.3×10e13 vg/mL, 4.4×10e13 vg/mL, 4.5×10e13 vg/mL, 4.6×10e13 vg/mL, 4.7×10e13 vg/mL, 4.8×10e13 vg/mL, 4.9×10e13 vg/mL, 5×10e13 vg/mL, 5.1×10e13 vg/mL, 5.2×10e13 vg/mL, 5.3×10e13 vg/mL, 5.4×10e13 vg/mL, 5.5×10e13 vg/mL, 5.6×10e13 vg/mL, 5.7×10e13 vg/mL, 5.8×10e13 vg/mL, 5.9×10e13 vg/mL, 6×10e13 vg/mL, 6.1×10e13 vg/mL, 6.2×10e13 vg/mL, 6.3×10e13 vg/mL, 6.4×10e13 vg/mL, 6.5×10e13 vg/mL, 6.6×10e13 vg/mL, 6.7×10e13 vg/mL, 6.8×10e13 vg/mL, 6.9×10e13 vg/mL, 7×10e13 vg/mL, 7.1×10e13 vg/mL, 7.2×10e13 vg/mL, 7.3×10e13 vg/mL, 7.4×10e13 vg/mL, 7.5×10e13 vg/mL, 7.6×10e13 vg/mL, 7.7×10e13 vg/mL, 7.8×10e13 vg/mL, 7.9×10e13 vg/mL, 8×10e13 vg/mL, 8.1×10e13 vg/mL, 8.2×10e13 vg/mL, 8.3×10e13 vg/mL, 8.4×10e13 vg/mL, 8.5×10e13 vg/mL, 8.6×10e13 vg/mL, 8.7×10e13 vg/mL, 8.8×10e13 vg/mL, 8.9×10e13 vg/mL, or 9×10e13 vg/mL. In other refinements, the retentate concentration is a range between any two concentrations provided above.

In various refinements, the TFF diafilters the AEX processed composition with a diavolume of 0 or more, 1 or more, 2 or more, 3 or more, 4 or more, 5 or more, 6 or more, 7 or more, 8 or more, 9 or more, 10 or more, 11 or more, 12 or more, 13 or more, 14 or more, or 15 of diafiltration to a TFF buffer.

The method and processes of various embodiments include processing the composition via TFF after ZUC processing. TFF includes the steps of ultrafiltration and diafiltration. This TFF step includes the steps of ultrafiltration and diafiltration, where the ZUC buffer including the gradient forming compound is removed from the composition and replaced with the AEX elution buffer is removed from the composition and replaced with a formulation buffer a including pharmaceutically acceptable carrier to prepare a pharmaceutical composition.

In various refinements, the ZUC processed composition loaded for TFF has a titer of at least 0.1×10e17 vg/ squared meter (m²), 0.1×10e17 vg/m², 0.5×10e17 vg/m², 1×10e17 vg/m², 1.5×10e17 vg/m², 2×10e17 vg/m², 2.5×10e17 vg/m², 3×10e17 vg/m², 3.5×10e17 vg/m², 4×10e17 vg/m², 4.5×10e17 vg/m², 5×10e17 vg/m², 5.5×10e17 vg/m², 6×10e17 vg/m², 6.5×10e17 vg/m², 7×10e17 vg/m², 7.5×10e17 vg/m², 8×10e17 vg/m², 8.5×10e17 vg/m², 9×10e17 vg/m², 9.5×10e17 vg/m 2 , or 10×10e17 vg/m 2 . In other refinements, the titer is a range between any two titers provided above.

In various refinements, the TFF filters the ZUC processed composition at a TMP of at least 2 psi (0.137895 bar), 2 psi (0.137895 bar), 3 psi (0.206843 bar), 4 psi (0.27579 bar), 5 psi (0.344738 bar), 6 psi (0.413685 bar), 7 psi (0.482633 bar), 8 psi (0.551581 bar), 9 psi (0.620528 bar), 10 psi (0.689476 bar), 11 psi (0.758423 bar), 12 psi (0.827371 bar), 13 psi (0.896318 bar), 14 psi (0.965266 bar), 15 psi (1.03421 bar), 16 psi (1.10316 bar), 17 psi (1.17211 bar), 18 psi (1.24106 bar), 19 psi (1.31 bar), 20 psi (1.37895 bar), 21 psi (1.4479 bar), 22 psi (1.51685 bar), 23 psi (1.58579 bar), 24 psi (1.65474 bar), 25 psi (1.72369 bar), 26 psi (1.79264 bar), 27 psi (1.86158 bar), 28 psi (1.93053 bar), 29 psi (1.99948 bar), 30 psi (2.06843 bar), 31 psi (2.13737 bar), 32 psi (2.20632 bar), 33 psi (2.27527 bar), 34 psi (2.34422 bar), 35 psi (2.41317 bar), 36 psi (2.48211 bar), 37 psi (2.55106 bar), 38 psi (2.62001 bar), 39 psi (2.68896 bar), 40 psi (2.7579 bar), 41 psi (2.82685 bar), 42 psi (2.8958 bar), 43 psi (2.96475 bar), 44 psi (3.03369 bar), 45 psi (3.10264 bar), 46 psi (3.17159 bar), 47 psi (3.24054 bar), 48 psi (3.30948 bar), 49 psi (3.37843 bar), or 50 psi (3.44738 bar). In other refinements, the TMP of the TFF for the ZUC processed composition is range between any two TMPs provided above.

In various refinements, the TFF filters the ZUC processed composition with a crossflow or retentate flow of at least 1 L/min/m², 1 L/min/m², 2 L/min/m², 3 L/min/m², 4 L/min/m², 5 L/min/m², 6 L/min/m², 7 L/min/m², 8 L/min/m², 9 L/min/m², 10 L/min/m², 11 L/min/m², 12 L/min/m², 13 L/min/m², 14 L/min/m 2 , or 15 L/min/m 2 . In other refinements, the crossflow of the TFF for the ZUC processed composition is a range between any two crossflows provided above.

In various refinements, the TFF filters the ZUC processed composition to a retentate concentration of at least 1×10e13 vg/mL, 1×10e13 vg/mL, 1.1×10e13 vg/mL, 1.2×10e13 vg/mL, 1.3×10e13 vg/mL, 1.4×10e13 vg/mL, 1.5×10e13 vg/mL, 1.6×10e13 vg/mL, 1.7×10e13 vg/mL, 1.8×10e13 vg/mL, 1.9×10e13 vg/mL, 2×10e13 vg/mL, 2.1×10e13 vg/mL, 2.2×10e13 vg/mL, 2.3×10e13 vg/mL, 2.4×10e13 vg/mL, 2.5×10e13 vg/mL, 2.6×10e13 vg/mL, 2.7×10e13 vg/mL, 2.8×10e13 vg/mL, 2.9×10e13 vg/mL, 3×10e13 vg/mL, 3.1×10e13 vg/mL, 3.2×10e13 vg/mL, 3.3×10e13 vg/mL, 3.4×10e13 vg/mL, 3.5×10e13 vg/mL, 3.6×10e13 vg/mL, 3.7×10e13 vg/mL, 3.8×10e13 vg/mL, 3.9×10e13 vg/mL, 4×10e13 vg/mL, 4.1×10e13 vg/mL, 4.2×10e13 vg/mL, 4.3×10e13 vg/mL, 4.4×10e13 vg/mL, 4.5×10e13 vg/mL, 4.6×10e13 vg/mL, 4.7×10e13 vg/mL, 4.8×10e13 vg/mL, 4.9×10e13 vg/mL, 5×10e13 vg/mL, 5.1×10e13 vg/mL, 5.2×10e13 vg/mL, 5.3×10e13 vg/mL, 5.4×10e13 vg/mL, 5.5×10e13 vg/mL, 5.6×10e13 vg/mL, 5.7×10e13 vg/mL, 5.8×10e13 vg/mL, 5.9×10e13 vg/mL, 6×10e13 vg/mL, 6.1×10e13 vg/mL, 6.2×10e13 vg/mL, 6.3×10e13 vg/mL, 6.4×10e13 vg/mL, 6.5×10e13 vg/mL, 6.6×10e13 vg/mL, 6.7×10e13 vg/mL, 6.8×10e13 vg/mL, 6.9×10e13 vg/mL, 7×10e13 vg/mL, 7.1×10e13 vg/mL, 7.2×10e13 vg/mL, 7.3×10e13 vg/mL, 7.4×10e13 vg/mL, 7.5×10e13 vg/mL, 7.6×10e13 vg/mL, 7.7×10e13 vg/mL, 7.8×10e13 vg/mL, 7.9×10e13 vg/mL, 8×10e13 vg/mL, 8.1×10e13 vg/mL, 8.2×10e13 vg/mL, 8.3×10e13 vg/mL, 8.4×10e13 vg/mL, 8.5×10e13 vg/mL, 8.6×10e13 vg/mL, 8.7×10e13 vg/mL, 8.8×10e13 vg/mL, 8.9×10e13 vg/mL, 9×10e13 vg/mL, 1×10e14 vg/mL, 1.1×10e14 vg/mL, 1.2×10e14 vg/mL, 1.3×10e14 vg/mL, 1.4×10e14 vg/mL, 1.5×10e14 vg/mL, 1.6×10e14 vg/mL, 1.7×10e14 vg/mL, 1.8×10e14 vg/mL, 1.9×10e14 vg/mL, or 2×10e14 vg/mL. In other refinements, the retentate concentration is a range between any two concentrations provided above.

In various refinements, the TFF diafilters the ZUC processed composition with a diavolume of 0 or more, 1 or more, 2 or more, 3 or more, 4 or more, 5 or more, 6 or more, 7 or more, 8 or more, 9 or more, 10 or more, 11 or more, 12 or more, 13 or more, 14 or more, or 15 of diafiltration to a to a TFF buffer.

Definitions

“Anion exchange chromatography” or “AEX” refers to processes separating an analyte from a mixture by flowing the mixture through an anion exchange material denoting an immobile matrix carrying covalently bound positively charged substituents. The “anion exchange material” is normally provided as an anion exchange chromatography column. The “anion exchange material” has the ability to exchange its not covalently bound counter ions for similarly charged binding partners or ions of the surrounding solution (e.g., mixture). Depending on the chemical nature of the charged group/substituent the “anion exchange material” can additionally be classified as strong or weak ion exchange material, depending on the strength of the covalently bound charged substituent. Strong anion exchange materials have a quartemary ammonium group, and weak anion exchange materials have a diethylaminoethyl group as charged substituent. Anion exchange chromatography includes the steps of equilibrating the column with a buffer, flowing the composition through the column, washing the column, and eluting of the composition from the column.

“Zonal ultracentrifugation” or “ZUC” refers to processes of centrifuging a composition using a zonal rotor. Examples of zonal rotors and zonal ultracentrifugation systems are disclosed in U.S. Pat. Nos. 6,051,189; 7,862,494; 7,837,609; 9,862,936; and 9,956,564, all of which are incorporated herein by reference in their entirety. One example of zonal ultracentrifugation is isopycnic density-gradient sedimentation, which relies on differences in the buoyant properties of the constituent particles dispersed in a high density solution as the basis for separation of the constituents.

“Tangential flow filtration” or “TFF” refers to an ultrafiltration process, where a solution containing capsids to be concentrated flows tangentially along the surface of an ultrafiltration filtration membrane. The filtration membrane has a pore size with a certain cut off value that prevents capsids from flowing through the filtration membrane as the permeate. Thus, the capsids are part of the retentate. Tangential flow filtration also includes diafiltration, where the original solution is removed as the permeate and is replaced with another solution. For example, tangential flow filtration replaces the elution buffer from the composition after anion exchange chromatography processing with the loading buffer for zonal ultracentrifugation processing. In another example, tangential flow filtration replaces the elution buffer from the composition after zonal ultracentrifugation processing with a formulation buffer a including a pharmaceutically acceptable carrier to prepare a pharmaceutical composition.

“Pharmaceutical product” refers to a product suitable for pharmaceutical use in a subject animal, including humans and mammals. For example, the pharmaceutical product is an rAAV virion.

“Pharmaceutical composition” refers to a composition suitable for pharmaceutical use in a subject animal, including humans and mammals. A pharmaceutical composition includes a pharmacologically effective amount of a pharmaceutical product, such as an AAV virion, and also includes a pharmaceutically acceptable carrier. A pharmaceutical composition encompasses a composition including the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions encompass any composition made by admixing a virion provided herein and a pharmaceutically acceptable carrier.

“Pharmaceutically acceptable carrier” refers to any of the standard pharmaceutical excipients, vehicles, diluents, stabilizers, preservatives, solubilizers, emulsifiers, adjuvants and/or carriers, such as, for example and not for limitation, a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Pharmaceutical carriers to be used can depend upon the intended mode of administration of the active agent. Typical modes of administration include enteral (e.g., oral) or parenteral (e.g., subcutaneous, intrathecal, intramuscular, intravenous or intraperitoneal injection; or topical, transdermal, or transmucosal administration). A “pharmaceutically acceptable salt” is a salt that can be formulated into an oxalate degrading enzyme composition for pharmaceutical use including, e.g., metal salts (sodium, potassium, magnesium, calcium, etc.) and salts of ammonia or organic amines.

“Pharmaceutically acceptable” or “pharmacologically acceptable” mean a material which is not biologically or otherwise undesirable, i.e., the material can be administered to an individual without causing any undesirable biological effects or interacting in a deleterious manner with any of the components of the composition in which it is contained.

“Subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

“Contaminating virus” refers to viruses that contaminate the composition during production processes. Contaminating virus impair the safety of the pharmaceutical product for administration into a subject. Examples of contaminating viruses include baculovirus such as Autographa californica nuclear polyhedrosis virus (AcNPV), encephalomyocarditis virus (EMC), porcine parvovirus (PPV), reovirus (Reo-3), simian vacuolating virus 40 (SV-40), vesicular stomatitis virus (VSV), or retroviruses such as murine leukemia virus (X-MuLV).

Adeno-Associated Virus

The therapeutically effective rAAV particles include rAAV particles disclosed in U.S. Pat. No. 9,504,762, WO 2019/222136, and US 2019/0376081, the disclosures of which are hereby incorporated in their entirety by reference.

“AAV” is a standard abbreviation for adeno-associated virus. Adeno-associated virus is a single-stranded DNA parvovirus having a genome encapsulated by a capsid. There are currently thirteen serotypes of AAV that have been characterized. General information and reviews of AAV can be found in, for example, Carter, 1989, Handbook of Parvoviruses, Vol. 1, pp. 169-228; and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, e.g., Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control.

An “AAV viral particle” as used herein refers to an infectious viral particle composed of at least one AAV capsid protein and an encapsidated AAV genome. “Recombinant AAV” or “rAAV”, “rAAV virion” or “rAAV viral particle” or “rAAV vector particle” or “AAV virus” refers to a viral particle composed of at least one capsid or Cap protein and an encapsidated rAAV vector genome as described herein. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of AAV vector particles necessarily includes production of rAAV vector, as such a vector is contained within an rAAV vector particle. The rAAV viral particle of different embodiments include AAV particles and rAAV particles disclosed in EP 2,698,163; EP 2,859,016; EP 3,044,231; EP 3,352,787; EP 3,491,008; EP 3,794,016; EP 3,794,112; U.S. Pat. Nos. 9,393,323; 9,447,168; 9,504,762; 9,764,045; 10,124,041; 10,463,718; 10,512,675; 10,709,796; 10,792,336; US 2017/0087219; US 2019/0376081; US 2020/0024579; US 2020/0061161; US 2020/0069819; US 2020/0362368; WO 2015/038625; WO 2017/053677; WO 2018/022608; WO 2019/217513; WO 2019/222132; WO 2019/222136; WO 2020/232044; WO 2021/097157; WO 2021/183895; and WO 2021/202943, the disclosures of which are hereby incorporated in their entirety by reference.

“Capsid” refers to the structure in which the rAAV vector genome is packaged. The capsid includes VP1 proteins or VP3 proteins, but more typically, all three of VP1, VP2, and VP3 proteins, as found in native AAV. The sequence of the capsid proteins determines the serotype of the rAAV virions. rAAV virions include those derived from a number of AAV serotypes, including AAV1, AAV2, AAV4, AAVS, AAV6, AAV7, AAV8, AAV9, AAV10, AAV11, AAV12, AAV13, is AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVCS, AAVF3, AAVFS, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV- LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2 , AAV Shuffle 100-1 , AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8 , AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAV_po.6, AAV_po., AAV_po.5, AAV_LK03, AAV_ra.1, AAV_bat_YNM, AAV_bat_Brazil, AAV_mo.1, AAV_avian_DA-1, or AAV_mouse_NY1, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpol, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, AAVanc80L65, or any variants thereof (see, e.g., U.S. Pat. No. 8,318,480 for its disclosure of non-natural mixed serotypes). Exemplary capsids are also provided in International Application Publication No. WO 2018/022608 and WO 2019/222136, which are incorporated herein in its entirety. The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. In various embodiments, the capsid of the AAV viral particle has an acetylated or unacetylated VP1, VP2, or VP3 protein with an amino acid sequence that is at least about 50%, 55%, 60%, 65%, 70%, 75%, 80%, 85%, 90%, 95%, 96%, 97%, 98%, 99% or 100% identical to a portion of an amino acid sequence from AAV-1 (Genbank Accession No. AAD27757.1), AAV-2 (NCBI Reference Sequence No. YP_680426.1), AAV-3 (NCBI Reference Sequence No. NP_043941.1), AAV-3B (Genbank Accession No. AAB95452.1), AAV-4 (NCBI Reference Sequence No. NP_044927.1), AAV-5 (NCBI Reference Sequence No. YP_068409.1), AAV-6 (Genbank Accession No. AAB95450.1), AAV-7 (NCBI Reference Sequence No. YP_077178.1), AAV-8 (NCBI Reference Sequence No. YP 077179.1), AAV-9 (Genbank Accession No. AAS99264.1), AAV-10 (Genbank Accession No. AAT46337.1), AAV-11 (Genbank Accession No. AAT46339.1), AAV-12 (Genbank Accession No. ABI16639.1), AAV-13 (Genbank Accession No. ABZ10812.1), or any amino acid sequence disclosed in WO 2018/022608 and WO 2019/222136. Construction and use of AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

“AAV vector”, “rAAV vector”, “vector genome”, and “rAAV vector genome” refer to nucleic acids, either single-stranded or double-stranded, having an AAV 5′ inverted terminal repeat (ITR) sequence and an AAV 3′ ITR flanking a protein-coding sequence (preferably a functional therapeutic protein-encoding sequence; e.g., FVIII, FIX, and PAH) operably linked to transcription regulatory elements that are heterologous to the AAV viral genome, i.e., one or more promoters and/or enhancers and, optionally, a polyadenylation sequence and/or one or more introns inserted between exons of the protein-coding sequence. The term “Gene of Interest” (GOI) can also refer to an rAAV vector genome. A single-stranded rAAV vector refers to nucleic acids that are present in the genome of an AAV virus particle and can be either the sense strand or the anti-sense strand of the nucleic acid sequences disclosed herein. The size of such single-stranded nucleic acids is provided in bases. A double-stranded rAAV vector refers to nucleic acids that are present in the DNA of plasmids, e.g., pUC19, or genome of a double-stranded virus, e.g., baculovirus, used to express or transfer the rAAV vector nucleic acids. The size of such double-stranded nucleic acids is provided in base pairs (bp). The term “ITR” as used herein refers to the art-recognized regions found at the 5′ and 3′ termini of the rAAV genome which function in cis as origins of DNA replication and as packaging signals for the viral genome. AAV ITRs, together with the Rep coding region, provide for efficient excision and rescue from the endosome, and integration of a nucleotide sequence interposed between two flanking ITRs into a host cell genome. Sequences of certain AAV-associated ITRs are disclosed by Yan et al., J. Virol. 79(1):364-379 (2005). ITRs are also found in a “flip” or “flop” configuration in which the sequence between the AA' inverted repeats (that form the arms of the hairpin) are present in the reverse complement (Willmott, Patrick. et al. Humon gene therapy methods 30.6 (2019): 206-213) Construction and use of AAV vector genomes of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001. Because of wide construct availability and extensive characterization, illustrative AAV vector genomes disclosed below are derived from serotype 2.

The terms “therapeutically effective AAV”, “therapeutically effective AAV particle”, “therapeutic AAV”, “therapeutically effective rAAV”, “therapeutically effective rAAV particle”, “therapeutic rAAV”, and “therapeutically effective rAAV” refer to recombinant AAV that are capable of infecting cells such that the infected cells express (e.g., by transcription and/or by translation) an element (e.g., nucleotide sequence, protein, etc.) of interest. To this extent, the therapeutically effective rAAV particles can include AAV particles having capsids or vector genomes (vgs) with different properties. For example, the therapeutically effective rAAV particles can have capsids with different post translation modifications. In other examples, the therapeutically effective AAV particles can contain vgs with differing sizes/lengths, plus or minus strand sequences, different flip/flop ITR configurations flip/flop, flop/flip, flip/flip, flop/flop, etc.), different number of ITRs (1, 2, 3, etc.), or truncations. For example, overlapping homologous recombination occurs in rAAV infected cells between nucleic acids having 5′ end truncations and 3′ end truncations so that a “complete” nucleic acid encoding the large protein is generated, thereby reconstructing a functional, full-length gene. In other examples, complementary nucleic acid sequences having 5′ end truncations and 3′ end truncations interact with each such that a “complete” nucleic acid is formed during second strand synthesis. The “complete” nucleic acid encodes the large protein, thereby reconstructing a functional, full-length gene. Therapeutically effective rAAV particles are also referred to as heavy capsids, full capsids, or partially full capsids.

The term “therapeutically effective amount” means an amount of a therapeutic agent that is sufficient, when administered to a subject suffering from or susceptible to a disease, disorder, or condition, to treat, diagnose, prevent, or delay the onset of the symptom(s) of the disease, disorder, or condition. It will be appreciated by those of ordinary skill in the art that a therapeutically effective amount is typically administered via a dosing regimen comprising at least one unit dose. The term “therapeutically effective” refers to any element or composition of a therapeutic agent acting sufficiently such that a therapeutically effective amount of the therapeutic agent is sufficient, when administered to a subject suffering from or susceptible to a disease, disorder, and/or condition, to treat, diagnose, prevent, and/or delay the onset of the symptom(s) of the disease, disorder, and/or condition. For example as previously noted, a therapeutically effective rAAV is capable of infecting cells such that the infected cells express (e.g., by transcription and/or by translation) an element (e.g., nucleotide sequence, protein, etc.) of interest. The therapeutically effective rAAV has a vector genome that is used by cells infected by the therapeutically effective rAAV to generate therapeutically effective nucleotide sequences that are used by the infected cell to generate an element (e.g., nucleotide sequence, protein, etc.) of interest by various methods such as replication, transcription, or translation. It is also noted that a “therapeutic agent” includes therapeutically effective rAAV or a therapeutic rAAV virus.

As an example, a “therapeutic rAAV virus”, which refers to an rAAV virion, rAAV viral particle, rAAV vector particle, or rAAV virus that comprises a heterologous polynucleotide that encodes a therapeutic protein, can be used to replace or supplement the protein in vivo. The “therapeutic protein” is a polypeptide that has a biological activity that replaces or compensates for the loss or reduction of activity of a corresponding endogenous protein. For example, a functional phenylalanine hydroxylase (PAH) is a therapeutic protein for phenylketonuria (PKU). Thus, for example recombinant rAAV PAH virus can be used for a medicament for the treatment of a subject suffering from PKU. The medicament may be administered by intravenous (IV) administration and the administration of the medicament results in expression of PAH protein in the bloodstream of the subject sufficient to alter the neurotransmitter metabolite or neurotransmitter levels in the subject. Optionally, the medicament may also comprise a prophylactic and/or therapeutic corticosteroid for the prevention and/or treatment of any hepatotoxicity associated with administration of the rAAV PAH virus. The medicament comprising a prophylactic or therapeutic corticosteroid treatment may comprise at least 5, 10, 15, 20, 25, 30, 35, 40, 45, 50, 55, 60, or more mg/day of the corticosteroid. The medicament comprising a prophylactic or therapeutic corticosteroid may be administered over a continuous period of at least about 3, 4, 5, 6, 7, 8, 9, 10 weeks, or more. The PKU therapy may optionally also include tyrosine supplements.

“Therapeutically ineffective AAV particle”, “therapeutically ineffective AAV”, “therapeutically ineffective rAAV particle”, or “therapeutically ineffective rAAV” refer to AAV particles that are incapable of infecting cells or a cell infected with therapeutically ineffective rAAV particles are unable to express (e.g., by transcription and/or by translation) an element (e.g., nucleotide sequence, protein, etc.) of interest. Therapeutically ineffective rAAV particles can contribute to decreased effectiveness per unit dose of capsid and can increase the risk of an immune response due to a needed increase of foreign proteins being introduced into the patient for an effective amount of heavy/full/partially full capsid. Therapeutically ineffective rAAV particles can include AAV particles having capsids or vgs with different properties and are referred to as empty capsids or light capsids. For example, empty capsids do not have a vg or have an unquantifiable or undetectable vg concentration. In another example, light capsids may have vgs with incomplete expression cassettes that do not express a gene of interest. In one example, the vector genomes of light capsids have one or more sizes that are insufficient for cells infected by the capsids to generate therapeutically effective nucleotide sequences. In another example, the light capsids the vector genomes of light capsids have one or more sizes that reduce expression of an element by a cell infected with the capsids and therapeutically effective rAAV encoding the element relative to expression of the element by a cell infected under the same conditions but being devoid of the infection with the capsid. In different examples, the size of a vector genomes of light capsid is 50% or less, 49% or less, 48% or less, 47% or less, 46% or less, 45% or less, 44% or less, 43% or less, 42% or less, 41% or less, 40% or less, 39% or less, 38% or less, 37% or less, 36% or less, 35% or less, 34% or less, 33% or less, 32% or less, 31% or less, 30% or less, 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, or 1% or less than the size of a vector genome of a therapeutically effective rAAV. Empty or light capsids can also have different capsid properties that can impair the infectivity of the capsids. In another example, therapeutically ineffective rAAV particles include rAAV particles having a Rep protein(s) associated with the particles. The rAAV associated with Rep protein(s) include, for example, large Rep proteins (e.g., Rep78 or Rep68 proteins), small Rep proteins (e.g., Rep52 or Rep40 proteins), or combinations thereof. The rAAV associated with Rep protein(s) can also include Rep protein(s) that are removed with capsids during different steps such as washing or regeneration steps in AEX processing or isolation of Post-pool fractions during ZUC processing. Alternatively, the rAAV associated with Rep protein(s) can also include large Rep proteins, small Rep proteins, or combinations thereof that are attached to capsids. Such attachments include, for example, different bonding stopes such as covalent bonding, ionic bonding, hydrogen/electrostatic bonding, or Van der Waals forces. In different examples, the Rep protein(s) can be attached to different parts of the rAAV particle including the capsid or vector genome when a portion of the vector genome is not encapsulated within the capsid. These capsids can be devoid of a vector genome or have a partial/full vector genome but are incapable of infecting cells. In another example, therapeutically ineffective rAAV particles include rAAV particles having deamidated capsids. For example, deamidated capsids include capsids having deamidated VP1, VP2, or VP3 proteins. For example, the conserved NG (Asp-Gly) residue in N-terminal region of the VP1 is vulnerable to deamidation. Different deamidated capsids and their effects on infectivity, transgene expression, or potency have been described by Giles, April R., et al. “Deamidation of Amino Acids on The Surface of Adeno-Associated Virus Capsids Leads to Charge Heterogeneity and Altered Vector Function.” Molecular Therapy 26.12 (2018): 2848-2862 and Frederick, Amy, et al. “Engineered Capsids for Efficient Gene Delivery to The Retina and Cornea” Human Gene Therapy 31.13-14 (2020): 756-774. While not being bound to by any particular theory, the heavy, full, or partially full capsids differ from light or empty capsids in their charge and/or density.

“AAV production impurities” refer to impurities that may impair the efficacy of the therapeutically effective rAAV. AAV production impurities occur during an rAAV preparation and include therapeutically ineffective rAAV, aggregates of the rAAV particles, extrinsic high molecular weight DNA, small nucleotides, proteins, buffer components, etc.

The transgene incorporated into the AAV capsid is not limited and may be any heterologous gene of therapeutic interest. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell.

The composition of the transgene sequence will depend upon the use to which the resulting vector will be put. For example, one type of transgene sequence includes a reporter sequence, which upon expression produces a detectable signal. Such reporter sequences include, without limitation, DNA sequences encoding b-lactamase, b-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

However, the transgene is typically a non-marker sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated animal. Typically, suitable target sequences include oncologic targets and viral diseases. See for examples of such targets the oncologic targets and viruses identified below in the section relating to immunogens.

The transgene may be used to correct or ameliorate gene deficiencies, which may include deficiencies in which normal genes are expressed at less than normal levels or deficiencies in which the functional gene product is not expressed. A preferred type of transgene sequence encodes a therapeutic protein or polypeptide which is expressed in a host cell. The vector may further include multiple transgenes, e.g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases (Kb). It is also noted that longer genomes (i.e., >5 (Kb)) might be feasible due to recombination of partial genomes in target cells. As an alternative to an IRES, the DNA may be separated by sequences encoding a 2A peptide, which self-cleaves in a post-translational event. See, e.g., Donnelly et al, J Gen. Virol., 78(Pt 1): 13-21 (January 1997); Furler, et al, Gene Ther., 8(11):864-873 (June 2001); Klump et al, Gene Ther., 8(10):8 11-817 (May 2001). This 2A peptide is significantly smaller than an IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

Suitable transgenes may be readily selected by one of skill in the art. The selection of the transgene is not considered to be a limitation of this invention. The transgene may be a heterologous protein, and this heterologous protein may be a therapeutic protein. Exemplary therapeutic proteins include, but are not limited to, blood factors, such as b-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a.), transforming growth factor beta (TGF-.b.), and the like; soluble receptors, such as soluble TNF-a. receptors, soluble VEGF receptors, soluble interleukin receptors (e.g., soluble IL-1 receptors and soluble type II IL-1 receptors), soluble g/d T cell receptors, ligand-binding fragments of a soluble receptor, and the like; enzymes, such as a-glucosidase, imiglucarase, b-glucocerebrosidase, and alglucerase; enzyme activators, such as tissue plasminogen activator; chemokines, such as 1P-10, monokine induced by interferon-gamma (Mig), Groa/IL-8, RANTES, MIP-1a, MIR-1b., MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors, luteinizing hormone; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IF-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; hereditary angioedema related proteins such as C1-inhibitor; dystrophin, mini-dystrophin, or microdystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GFUT2), aldolase A, b-enolase, and glycogen synthase; lysosomal enzymes (e.g., beta-N-acetylhexosaminidase A); and any variants thereof. Other transgenes include transgenes encoding cardiac myosin binding protein C, β-myosin heavy chain, cardiac troponin T, cardiac troponin I, myosin ventricular essential light chain 1, myosin ventricular regulatory light chain 2, cardiac a actin (ACTC), α-tropomyosin, titin, four-and-a-half LIM protein 1, and other transgenes disclosed in U.S. Patent No. in International Application Publication No. WO 2014/170470. The AAV vector also includes conventional control elements or sequences which are operably linked to the transgene in a manner which permits its transcription, translation and/or expression in a cell transfected with the plasmid vector or infected with the virus. As used herein, “operably linked” sequences include both expression control sequences that are contiguous with the gene of interest and expression control sequences that act in trans or at a distance to control the gene of interest. Suitable genes include those genes discussed in Anguela et al. “Entering the Modern Era of Gene Therapy “, Annual Rev. of Med. Vol. 70, pages 272-288 (2019) and Dunbar et al., “Gene Comes of Age”, Science, Vol. 359, Issue 6372, eaan4672 (2018).

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart el al, Cell, 41:521-530 (1985)), the SV40 promoter, the dihydrofolate reductase promoter, the b-actin promoter, the phosphoglycerol kinase (PGK) promoter, and the EF1 promoter [Invitrogen]. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracyclinerepressible system [Gossen et al., Proc. Natl. Acad. Sci. USA, 89:5547-5551 (1992)], the tetracycline-inducible system [Gossen et al., Science, 268:1766-1769 (1995), see also Harvey et al., Curr. Opin. Chem. Biol., 2:512-518 (1998)1, the RU486-inducible system [Wang et al., Nat. Biotech., 15:239-243 (1997) and Wang et al., Gene Ther., 4:432-441 (1997)] and the rapamycininducible system [Magari et al., J. Clin. Invest., 100:2865-2872 (1997)]. Other types of inducible promoters which may be useful in this context are those which are regulated by a specific physiological state, e.g., temperature, acute phase, a particular differentiation state of the cell, or in replicating cells only.

Optionally, the native promoter for the transgene may be used. The native promoter may be preferred when it is desired that expression of the transgene should mimic the native expression. The native promoter may be used when expression of the transgene must be regulated temporally or developmentally, or in a tissue- specific manner, or in response to specific transcriptional stimuli. In a further embodiment, other native expression control elements, such as enhancer elements, polyadenylation sites or Kozak consensus sequences may also be used to mimic the native expression.

The transgene may also include a gene operably linked to a tissue specific promoter. For instance, if expression in skeletal muscle is desired, a promoter active in muscle should be used. These include the promoters from genes encoding skeletal b-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

The recombinant AAV can be used to produce a protein of interest in vitro, for example, in a cell culture. For example, the AAV can be used in a method for producing a protein of interest in vitro, where the method includes providing a recombinant AAV comprising a nucleotide sequence encoding the heterologous protein; and contacting the recombinant AAV with a cell in a cell culture, whereby the recombinant AAV expresses the protein of interest in the cell. The size of the nucleotide sequence encoding the protein of interest can vary. For example, the nucleotide sequence can be at least about 0.1 kilobases (kb), at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide is at least about 1.4 kb in length.

The recombinant AAV can also be used to produce a protein of interest in vivo, for example in an animal such as a mammal. Some embodiments provide a method for producing a protein of interest in vivo, where the method includes providing a recombinant AAV comprising a nucleotide sequence encoding the protein of interest; and administering the recombinant AAV to the subject, whereby the recombinant AAV expresses the protein of interest in the subject. The subject can be, in some embodiments, a non-human mammal, for example, a monkey, a dog, a cat, a mouse, or a cow. The size of the nucleotide sequence encoding the protein of interest can vary. For example, the nucleotide sequence can be at least about 0.1 kb, at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide is at least about 1.4 kb in length.

Of particular interest is the use of recombinant AAV to express one or more therapeutic proteins to treat various diseases or disorders. Non-limiting examples of the diseases include cancer such as carcinoma, sarcoma, leukemia, lymphoma; and autoimmune diseases such as multiple sclerosis. Non-limiting examples of carcinomas include esophageal carcinoma; hepatocellular carcinoma; basal cell carcinoma, squamous cell carcinoma (various tissues); bladder carcinoma, including transitional cell carcinoma; bronchogenic carcinoma; colon carcinoma; colorectal carcinoma; gastric carcinoma; lung carcinoma, including small cell carcinoma and non-small cell carcinoma of the lung; adrenocortical carcinoma; thyroid carcinoma; pancreatic carcinoma; breast carcinoma; ovarian carcinoma; prostate carcinoma; adenocarcinoma; sweat gland carcinoma; sebaceous gland carcinoma; papillary carcinoma; papillary adenocarcinoma; cystadenocarcinoma; medullary carcinoma; renal cell carcinoma; ductal carcinoma in situ or bile duct carcinoma; choriocarcinoma; seminoma; embryonal carcinoma; Wilm's tumor; cervical carcinoma; uterine carcinoma; testicular carcinoma; osteogenic carcinoma; epithelieal carcinoma; and nasopharyngeal carcinoma. Non-limiting examples of sarcomas include fibrosarcoma, myxosarcoma, liposarcoma, chondrosarcoma, chordoma, osteogenic sarcoma, osteosarcoma, angiosarcoma, endothelio sarcoma, lymphangiosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Non-limiting examples of leukemias include chronic myeloproliferative syndromes; acute myelogenous leukemias; chronic lymphocytic leukemias, including B-cell CLL, T-cell CLL prolymphocytic leukemia, and hairy cell leukemia; and acute lymphoblastic leukemias. Examples of lymphomas include, but are not limited to, B-cell lymphomas, such as Burkitt's lymphoma; Hodgkin's lymphoma; and the like.

Other non-liming examples of the diseases that can be treated using rAAV and methods disclosed herein include genetic disorders including sickle cell anemia, cystic fibrosis, lysosomal acid lipase (LAL) deficiency 1, Tay-Sachs disease, Phenylketonuria, Mucopolysaccharidoses, Glycogen storage diseases (GSD, e.g., GSD types I, II, III, IV, V, VI, VII, VIII, IX, X, XI, XII, XIII, and XIV), Galactosemia, muscular dystrophy (e.g., Duchenne muscular dystrophy), cardiomyopathies (e.g., hypertrophic cardiomyopathy, dilated cardiomyopathy, arrhythmogenic right ventricular cardiomyopathy, etc.) and hemophilia such as hemophilia A (classic hemophilia) and hemophilia B (Christmas Disease), Wilson's disease, Fabry Disease, Gaucher Disease hereditary angioedema (HAE), and alpha 1 antitrypsin deficiency. In addition, the rAAV and methods disclosed herein can be used to treat other disorders that can be treated by local expression of a transgene in the liver or by expression of a secreted protein from the liver or a hepatocyte.

The amount of the heterologous protein expressed in the subject (e.g., the serum of the subject) can vary. For example, in some embodiments the protein can be expressed in the serum of the subject in the amount of at least about 9 milligram (mg)/mL, at least about 10 mg/mL, at least about 11 mg/mL, at least about 12 mg/mL, at least about 13 mg/mL, at least about 14 mg/mL, at least about 15 mg/mL, at least about 16 mg/mL, at least about 17 mg/mL, at least about 18 mg/mL, at least about 19 mg/mL, at least about 20 mg/mL, at least about 21 mg/mL, at least about 22 mg/mL, at least about 23 mg/mL, at least about 24 mg/mL, at least about 25 mg/mL, at least about 26 mg/mL, at least about 27 mg/mL, at least about 28 mg/mL, at least about 29 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 41 mg/mL, at least about 42 mg/mL, at least about 43 mg/mL, at least about 44 mg/mL, at least about 45 mg/mL, at least about 46 mg/mL, at least about 47 mg/mL, at least about 48 mg/mL, at least about 49 mg/mL, or at least about 50 mg/mL. The protein of interest may be expressed in the serum of the subject in the amount of about 9 pg/mL, about 10 pg/mL, about 50 pg/mL, about 100 pg/mL, about 200 pg/mL, about 300 pg/mL, about 400 pg/mL, about 500 pg/mL, about 600 pg/mL, about 700 pg/mL, about 800 pg/mL, about 900 pg/mL, about 1000 pg/mL, about 1500 pg/mL, about 2000 pg/mL, about 2500 pg/mL, or a range between any two of these values. A skilled artisan will understand that the expression level in which a protein of interest is needed for therapeutic efficacy can vary depending on non-limiting factors, such as the particular protein of interest and the subject receiving the treatment, and an effective amount of the protein can be readily determined by a skilled artisan using conventional methods known in the art without undue experimentation.

Methods of Producing Adeno-Associated Virus

Any method known in the art may be used for the preparation of a novel rAAV viral particle of the disclosure. In some embodiments, a novel rAAV viral particle is produced in mammalian cells (e.g., HEK293). In some embodiments, a novel rAAV viral particle is produced in insect cells (e.g., Sf9). In some embodiments, an AAV viral particle is prepared by providing to a host cell with an AAV genome vector comprising a transgene together with a Rep and Cap gene. In some embodiments, an AAV genome vector comprises a transgene, an AAV Rep gene and an AAV Cap gene. In some embodiments, an rAAV viral particle is prepared by providing to a host cell with two or more vectors. For example, in some embodiments, an AAV genome vector comprising a transgene is introduced (e.g., transfected or transduced) into a cell with a vector (e.g., a plasmid or baculovirus) comprising an AAV Rep gene and a AAV Cap gene. In some embodiments, a cell transfected or transduced with an AAV genome vector comprising a transgene, a vector (e.g., a plasmid or baculovirus) comprising an AAV Rep gene, and a vector (e.g., a plasmid or baculovirus) comprising an AAV Cap gene.

Methods of making AAV viral particles are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, WO2001023597, WO2015191508, WO2019217513, WO2018022608, WO2019222136, WO2020232044, WO2019222132; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88: 4646-50 (1991); Ruffing et al., J. Vir.66:6922-30 (1992); Kimbauer et al., Vir., 219:37-44 (1996); Zhao et al., Vir.272:382-93 (2000); the contents of each of which are herein incorporated by reference in their entirety.

Cells such as, e.g., an insect cell, yeast cell, and mammalian cell (e.g., human cell or non-human mammalian cell) are capable of generating rAAV. For example, cells are capable of generating rAAV when provided AAV helper functions, AAV non-helper functions, and a nucleotide sequence that the cells use to generate an AAV vector genome. In various embodiments, the AAV helper functions, AAV non-helper functions, and a nucleotide sequence that the cells use to generate rAAV are provided by a vector that is delivered to cell, for example, via transfection with transfection reagents, via transductions/infections with other recombinant viruses, by incorporating nucleotide sequences into the genomes of the cells, or by other methods. Examples of such cells include mammalian cell lines such as HEK293, HeLa, CHO, NS0, SP2/0, PER.C6, Vero, RD, BHK, HT 1080, A549, Cos-7, ARPE-19, and MRC-5 cells. In other examples, the insect cell line used can be from Spodoptera frugiperda, such as Sf9, SF21, SF900+, drosophila cell lines, mosquito cell lines, e.g., Aedes albopictus derived cell lines, domestic silkworm cell lines, e.g. Bombyx mori cell lines, Trichopiusia ni cell lines such as High Five cells or Lepidoptera cell lines, such as Ascalapha odorata cell lines. Preferred insect cells are cells from the insect species which are susceptible to baculovirus infection, including High Five, Sf9, Sf-RVN, Se301, SeIZD2109, SeUCR1, Sf900+, Sf21, BTI-TN-5B1-4, MG-1, Tn368, HzAml, BM-N, Ha2302, Hz2E5 and Ao38.

As previously described, the term “vector” is understood to refer to any genetic element, such as a plasmid, phage, transposon, cosmid, bacmid, mini-plasmid (e.g., plasmid devoid of bacterial elements), Doggybone DNA (e.g., minimal, closed-linear constructs), chromosome, virus, virion (e.g., baculovirus), etc., which is capable of replication when associated with the proper control elements and which can transfer gene sequences between cells. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. Baculoviral vectors and methods for their use are described in the above cited references on molecular engineering of insect cells.

The vector from which the cell generates an rAAV vector genome may contain a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. The vector may also contain a posttranscriptional regulatory element downstream of the restriction site and upstream of the 3′ AAV ITR. The viral construct may further comprise a polynucleotide inserted at the restriction site and operably linked with the promoter, where the polynucleotide comprises the coding region of a protein of interest.

The term “AAV helper” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The capsid (Cap) expression products supply necessary packaging functions. AAV helper functions are used herein to complement AAV functions in trans that are missing from AAV vector genomes.

In various embodiments, a vector providing AAV helper functions includes a nucleotide sequence(s) that encode capsid proteins or Rep proteins. The cap genes and/or rep gene from any AAV serotype (including, but not limited to, AAV1 (NCBI Reference Sequence No./Genbank Accession No. NC_002077.1), AAV2 (NCBI Reference Sequence No./Genbank Accession No. NC_001401.2), AAV3 (NCBI Reference Sequence No./Genbank Accession No. NC_001729.1), AAV3B (NCBI Reference Sequence No./Genbank Accession No. AF028705.1), AAV4 (NCBI Reference Sequence No./Genbank Accession No. NC_001829.1), AAVS (NCBI Reference Sequence No./Genbank Accession No. NC_006152.1), AAV6 (NCBI Reference Sequence No./Genbank Accession No. AF028704.1), AAV7 (NCBI Reference Sequence No./Genbank Accession No. NC_006260.1), AAV8 (NCBI Reference Sequence No./Genbank Accession No. NC_006261.1), AAV9 (NCBI Reference Sequence No./Genbank Accession No. AX753250.1), AAV10 (NCBI Reference Sequence No./Genbank Accession No. AY631965.1), AAV11 (NCBI Reference Sequence No./Genbank Accession No. AY631966.1), AAV12 (NCBI Reference Sequence No./Genbank Accession No. DQ813647.1), AAV13 (NCBI Reference Sequence No./Genbank Accession No. EU285562.1), is AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh. 54, AAV4-19/rh. 55, AAVS-3/rh.57, AAV5 -22/rh. 58, AAV7. 3/hu.7, AAV16. 8/hu.10, AAV16.12/hu.11, AAV29.3/bb. 1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33. 8/hu.16, AAV52/hu.19, AAV52.1/hu.20, AAV58.2/hu.25, AAVA3.3, AAVA3.4, AAVA3.5, AAVA3.7, AAVC1, AAVC2, AAVCS, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54,

AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV- LK19, AAV-PAEC2, AAV-PAEC4, AAV-PAEC6, AAV-PAEC7, AAV-PAEC8, AAV-PAEC11, AAV-PAEC12, AAV-2-pre-miRNA-101 , AAV-8h, AAV-8b, AAV-h, AAV-b, AAV SM 10-2 , AAV Shuffle 100-1 , AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAV_po.6, AAV_po., AAV_po.5, AAV_LK03, AAV_ra.1, AAV_bat_YNM, AAV_bat_Brazil, AAV_mo.1, AAV avian DA-1, or AAV mouse NY1, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpol, Bpo2, Bpo3, Bpo4, Bpo6, Bpo8, Bpol3, Bpol8, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, AAVanc80L65, or any variants thereof) can be used herein to produce the recombinant AAV Exemplary capsids are also provided in International Application No. WO 2018/022608 and WO 2019/222136, which are incorporated herein in its entirety. Each NCBI Reference Sequence Number or Genbank Accession Numbers provided above is also incorporated by reference herein. In some embodiments, the AAV cap genes encode a capsid from serotype 1, serotype 2, serotype 3, serotype 3B, serotype 4, serotype 5, serotype 6, serotype 7, serotype 8, serotype 9, serotype 10, serotype 11, serotype 12, serotype 13, or a variant thereof.

For production, cells with AAV helper functions produce recombinant capsid proteins sufficient to form a capsid. This includes at least VP1 and VP3 proteins, but more typically, all three of VP1, VP2, and VP3 proteins, as found in native AAV. The sequence of the capsid proteins determines the serotype of the AAV virions produced by the host cell. Capsids useful in the invention include those derived from a number of AAV serotypes, including 1, 2, 3, 3B, 4, 5, 6, 7, 8, 9, 10, 11, 12, 13 or mixed serotypes (see, e.g., U.S. Pat. No. 8,318,480 for its disclosure of non-natural mixed serotypes). The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. Because of wide construct availability and extensive characterization, illustrative AAV vectors disclosed below are derived from serotype 2. Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

In various embodiments, nucleotide sequences encoding VP proteins can be operably linked to a suitable expression control sequence. In various embodiments, nucleotide sequences encoding Rep proteins can be operably linked to a suitable expression control sequence such as eukaryotic promoters. For example, the nucleotide sequences can be operably linked to eukaryotic promoters. In another example, the nucleotide sequences can be operably linked to baculoviral promoters such as the polyhedrin (Polh) promoter, ΔIE1 promoter, p5 promoter, p10 promoter, the p40 promoter, metallothionein promoter, 39K promoter, p6.9 promoter, and orf46 promoter.

For production, cells with AAV helper functions produce Rep proteins to promote production of rAAV. It has been found that infectious particles can be produced when at least one large Rep protein (Rep78 or Rep68) and at least one small Rep protein (Rep52 and Rep40) are expressed in cells. In a specific embodiment all four of Rep 78, Rep68, Rep52 and Rep 40 are expressed. Alternately, Rep78 and Rep52, Rep78 and Rep40, Rep 68 and Rep52, or Rep68 and Rep40 are expressed. Examples below demonstrate the use of the Rep78/Rep52 combination. Rep proteins can be derived from AAV-2 or other serotypes. In various embodiments, nucleotide sequences encoding Rep proteins can be operably linked to a suitable expression control sequence. In various embodiments, nucleotide sequences encoding Rep proteins can be operably linked to a suitable expression control sequence such as eukaryotic promoters. For example, the nucleotide sequences can be operably linked to eukaryotic promoters. In other examples, the nucleotide sequences can be operably linked to baculoviral promoters such as the polyhedrin (Polh) promoter, ΔIE1 promoter, p5 promoter, p10 promoter, the p40 promoter, metallothionein promoter, 39K promoter, p6.9 promoter, and orf46 promoter.

Cells with AAV helper functions can also produce assembly-activating proteins (AAP), which help assemble capsids. In various embodiments, nucleotide sequences encoding AAP can be operably linked to a suitable expression control sequence. For example, the nucleotide sequences can be operably linked to eukaryotic promoters. In other examples, the nucleotide sequences can be operably linked to baculoviral promoters such as the polyhedrin (Polh) promoter, ΔIE1 promoter, p5 promoter, p10 promoter, the p40 promoter, metallothionein promoter, 39K promoter, p6.9 promoter, and orf46 promoter.

The term “non-AAV helper function” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

The term “non-AAV helper function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon or cosmid. In particular, it has been demonstrated that the full-complement of adenovirus genes are not required for accessory helper functions. For example, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982). J. Virol. 41:868; Janik et al., (1981) Proc. Natl. Acad. Sci. USA 78:1925; Carter et al., (1983) Virology 126:505. Other characterized Ad mutants include: EIB (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Although studies of the accessory functions provided by adenoviruses having mutations in the E1B coding region have produced conflicting results, Samulski et al., (1988) J. Virol. 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes. Particularly preferred accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

Host cells commonly used for production of rAAV viral particles include, but are not limited to, HEK293 cells, COS cells, HeLa cells, KB cells, and other mammalian cell lines as described in U.S. Pat. Nos. 6,156,303, 5,387,484, 5,741,683, 5,691,176, and 5,688,676; U.S. Patent Application Publication No. 2002/0081721, and International Patent Publication Nos. WO 2000047757, WO 2000024916, and WO 1996017947, the contents of each of which are herein incorporated by reference in their entirety. In some embodiments, the HEK293 cells may be HEK-293T cells. Other examples of mammalian cells that may be used for the production of AAV viral particles include A549, WEH1, 3T3, BHK, MDCK, COS 1, COS 7, BSC 1, BSC 40, BMT 10, VERO, W138, Saos, C2C12, L cells, HT1080, HepG2 and primary fibroblast, hepatocyte and myoblast cells derived from mammals. In some embodiments, host cells used for the production of AAV viral particles are cells derived from mammalian species including, but not limited to, human, monkey, mouse, rat, rabbit, and hamster. In some embodiments, host cells used for the production of AAV viral particles are cells derived from a cell type, including but not limited to fibroblast, hepatocyte, tumor cell, cell line transformed cell, etc.Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. (1989) vol. 63, pp.3822-3828; Kajigaya et al., Proc. Nat'l. Acad. Sci. USA (1991) vol. 88, pp. 4646-4650; Ruffing et al., J. Vir. (1992) vol. 66, pp. 6922-6930; Kirnbauer et al., Vir. (1996) vol. 219, pp. 37-44; Zhao et al., Vir. (2000) vol. 272, pp. 382-393; and U.S. Pat. No. 6,204,059). Examples of insect cell lines that can be used may be derived from Spodoptera frugiperda, such as Sf9, Sf21, Sf900+, drosophila cell lines, mosquito cell lines, e.g., Aedes albopictus derived cell lines, domestic silkworm cell lines, e.g., Bombyxmori cell lines, Trichoplusia ni cell lines such as High Five cells or Lepidoptera cell lines such as Ascalapha odorata cell lines. Exemplary insect cells are cells from the insect species which are susceptible to baculovirus infection, including High Five, Sf9, Sf-RVN, Se301, SeIZD2109, SeUCR1, Sf900+, Sf21, BTI-TN-5B1-4, MG-1, Tn368, HzAml, BM-N, Ha2302, Hz2E5 and Ao38.

In some embodiments, a novel rAAV viral particle is produced in an insect cell. Growing conditions for insect cells in culture, and production of heterologous products in insect cells in culture are well-known in the art, see U.S. Pat. No. 6,204,059, the contents of which are herein incorporated by reference in its entirety.

In various embodiments, insect cells having vectors for rAAV production are provided. Recombinant baculovirus (rBV) with nucleotide sequences for rAAV production can be used to deliver these nucleotide sequences to the insect cells for rAAV production. Baculoviruses, such as rBV, are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori nucleopolyhedrovirus (Bm-NPV) (Katou, Yasuhiro, et al., Virology 404.2 (2010): 204-214.). Baculoviruses are commonly used for the infection of insect cells for the expression of recombinant proteins. In particular, expression of heterologous genes in insects can be accomplished as described in for instance U.S. Pat. No. 4,745,051; Friesen, P. D., and L. K. Miller., Current topics in microbiology and immunology 131 (1986): 31-49; EP 127839; EP 155476; Vlak, Just M., et al., Journal of General Virology 69.4 (1988): 765-776; Miller, Lois K., Annual Reviews in Microbiology 411 (1988): 177-199; Carbonell, Luis F., et al., Gene 73.2 (1988): 409-418; Maeda, Susumu, et al., Nature 315,6020 (1985): 592-594; Lebacq-Verheyden, ANNE-MAR1E, et al. Molecular and cellular biology 8.8 (1988): 3129-3135; Smith, Gale E., et al., Proceedings of the National Academy of Sciences 82.24 (1985): 8404-8408; Miyajima, Atsushi, et al., Gene 58.2-3 (1987): 273-281; and Martin, Brian M., et al., DNA 7.2 (1988): 99-106. Numerous baculovirus strains and variants and corresponding permissive insect host cells that can be used for protein production are described in Luckow, Verne A., and Max D. Summers., Bio/technology 6.1 (1988): 47-55; Miller et al (1986) Genetic Engineering, Principles and Methods, Vol. 8 (eds. J. Setlow and A. Hollaender), Plenum Press, N.Y, 277-298, 1986); Maeda, Susumu, et al., Nature 31.5.6020 (1985): 592-594; and McKenna, Kevin A., Huazhu Hong, and Robert R. Granados, Journal of Invertebrate Pathology 71.1 (1998): 82-90.

A donor vector and a bacmid or a transfer vector and linearized baculovirus DNA are used for generating recombinant baculoviruses (rBV). Bacmids propagate in bacteria such as Escherichia coli as a large plasmid. When transfected into insect cells, the bacmids generate baculovirus. Traditional baculovirus generation, e.g. as is one in the Invitrogen's Bac-to-Bac system generates recombinant baculovirus by site-specific transposition in E. coli. High molecular weight bacmid DNA is then isolated and transfected into Sf9 or Sf21 cells from which recombinant baculovirus is isolated and amplified.

Insect cells can be separately transfected with bacmids having nucleotide sequences for rAAV vector genome or having nucleotide sequences providing AAV helper functions to generate rBV. These different rBVs are subsequently used to co-infect naive insect cells to generate rAAV.

In various embodiments, the cells after transfection are cultured for about 12 hours, about 24 hours, about 36 hours, about 48 hours, about 72 hours, about 96 hours, about 120 hours, about 144 hours, about 168 hours, about 192 hours, about 216 hours, about 240 hours, or a time between any of these two time points after transfection.

As previously noted, the increased concentrations of therapeutically effective rAAV particles are an economically viable quantity that can be processed each time by ZUC. In one example, the process of various embodiments can allow for greater concentrations of rAAV to be produced such that larger cell culture volumes can be used for rAAV production. For example, host cells capable of producing rAAV can be cultured in a volume of at least 5 milliliters (mL), at least 10 mL, at least 20 mL, at least 50 mL, at least 100 mL, at least 500 mL, at least 1 liter (L), at least 10 L, at least 50 L, at least 100L, at least 250 L, at least 500 L, at least 1000 L, at least 1500 L, at least 2000 L, or at least 2500 L. Culturing can also occur in a spin tube(s), a shake flask(s), or a bioreactor(s).

Clarification: To remove rAAV virions from cultured host cells, a number of methods can be employed. In one example, the cells are lysed and the virus can be purified. Alternatively, the virus is expressed into the supernatant. The rAAV virions can be purified by centrifugation, filtration, tangential flow filtration, chromatography, or a combination thereof.

In one example of such a method, the insect cells are resuspended in lysis buffer (20 mM Tris-Cl pH=8, 150 mM NaCl, 0.5% deoxychloate), and lysed using glass beads. The lysate is treated with Benzonase (Sigma, St. Louis, Mo.) and centrifuged at 4000 g and the supernatant is chromatographed on Streamline HE column (Pharmacia), Phenyl Sepharose, and POROS HE (Potter et al., Methods Enzymol 346:413-30, 2002).

Encapsidation/Infectivity: To assess vector genome encapsidation, the purified AAV virion can be treated with nuclease to degrade any non-encapsidated DNA. The encapsidated DNA will be protected from the nuclease and thus be detectable after the nuclease treatment. The Examples below demonstrate vector genomes that survive Benzonase treatment, as determined by Southern blotting.

To assess virion performance, the purified rAAV virions are used to infect HEK293 cells in culture or are injected into mouse skeletal muscle to assess their infectivity by scoring for cells expressing GFP. Where the payload gene is not optically accessible, other detection techniques such as Western blotting, immunoassay, PCR, or reverse transcription PCR, or functional assay can be employed to assess infectivity. In Example 3 of U.S. Patent Publication No. 2015/0071883, immunoassay and coagulation assays are used to assess transduction of Rag2 mice with Factor VIII-expressing rAAV. Thus, the measure of infectivity can vary depending on the payload gene and model system.

Generally, the virions are infectious such that upon incubating a HEK293 cell in the presence of a virion solution containing 10⁷ (10\hat{ }7, 1E07) viral genomes, the exogenous gene is expressed in detectable amounts by the cell. Alternately, the virions are infectious such that upon incubating a HEK293 cell in the presence of a virion solution containing 10\hat{ }6 viral genomes, the exogenous gene is expressed in detectable amounts by the cell.

Formulation: Various formulations of rAAV are known in the art. Purified rAAV can be diluted or dialyzed into saline with optional buffer, carrier, and/or stabilizer. Known AAV formulations include those using polaxamer, PEG, sugar, polyhydric alcohols, or multivalent ion salts. See, e.g., U.S. Pat. Nos. 8,852,607 and 7,704,721. An exemplary formulation is 1.38 mg/ml sodium phosphate, monobasic monohydrate, 1.42 mg/ml sodium phosphate, dibasic (dried), 8.18 mg/ml sodium chloride, 20 mg/ml mannitol and 2.0 mg/ml Poloxamer 188 (Pluronic F-68), pH 7.4.

In other examples, the rAAV pharmaceutical formulation of the invention comprises one or more pharmaceutically acceptable excipients to provide the formulation with advantageous properties for storage and/or administration to subjects for treatment. In certain embodiments, the pharmaceutical formulations of the present invention are capable of being stored at <65° C. for a period of at least 2 weeks, preferably at least 4 weeks, more preferably at least 6 weeks and yet more preferably at least about 8 weeks, without detectable change in stability. In this regard, the term “stable” means that the rAAV present in the formulation essentially retains its physical stability, chemical stability and/or biological activity during storage. In certain embodiments of the present invention, the recombinant AAV virus present in the pharmaceutical formulation retains at least about 80% of its biological activity in a human patient during storage for a determined period of time at -65° C., more preferably at least about 85%, 90%, 95%, 98% or 99% of its biological activity in a human patient.

In various examples, the formulation comprising rAAV further comprises one or more buffering agents. Other buffering agents are disclosed in Sek, D. “Breaking old habits: moving away from commonly used buffers in pharmaceuticals.” European Pharmaceutical Review 3 (2012). For example, the formulation of the present invention comprises sodium phosphate dibasic at a concentration of about 0.1 mg/ml to about 3 mg/ml, about 0.5 mg/ml to about 2.5 mg/ml, about 1 mg/ml to about 2 mg/ml, or about 1.4 mg/ml to about 1.6 mg/ml. In a particularly preferred embodiment, the rAAV formulation of the present invention comprises about 1.42 mg/ml of sodium phosphate, dibasic (dried). Another buffering agent that may find use in the rAAV formulations of the present invention is sodium phosphate, monobasic monohydrate which, in some embodiments, finds use at a concentration of from about 0.1 mg/ml to about 3 mg/ml, about 0.5 mg/ml to about 2.5 mg/ml, about 1 mg/ml to about 2 mg/ml, or about 1.3 mg/ml to about 1.5 mg/ml. In a particularly preferred embodiment, the rAAV formulation of the present invention comprises about 1.38 mg/ml of sodium phosphate, monobasic monohydrate. In a yet more particularly preferred embodiment of the present invention, the rAAV formulation of the present invention comprises about 1.42 mg/ml of sodium phosphate, dibasic and about 1.38 mg/ml of sodium phosphate, monobasic monohydrate.

In another aspect, the rAAV formulation of the present invention may comprise one or more isotonicity agents, such as sodium chloride, preferably at a concentration of about 1 mg/ml to about 20 mg/ml, for example, about 1 mg/ml to about 10 mg/ml, about 5 mg/ml to about 15 mg/ml, or about 8 mg/ml to about 20 mg/ml. In a particularly preferred embodiment, the formulation of the present invention comprises about 8.18 mg/ml sodium chloride. Other buffering agents and isotonicity agents known in the art are suitable and may be routinely employed for use in the formulations of the present disclosure.

In another aspect, the rAAV formulations of the present invention may comprise one or more bulking agents. Exemplary bulking agents include without limitation mannitol, sucrose, dextran, lactose, trehalose, and povidone (PVP 1(24). In certain preferred embodiments, the formulations of the present invention comprise mannitol, which may be present in an amount from about 5 mg/ml to about 40 mg/ml, or from about 10 mg/ml to about 30 mg/ml, or from about 15 mg/ml to about 25 mg/ml. In a particularly preferred embodiment, mannitol is present at a concentration of about 20 mg/ml.

In yet another aspect, the rAAV formulations of the present invention may comprise one or more surfactants, which may be non-ionic surfactants.

Other aspects and advantages of the present disclosure will be understood upon consideration of the following illustrative examples.

### EXEMPLIFIED EMBODIMENTS OF THE INVENTION

### Example 1

### Impact of Light Capsids on Infectivity and Transgene Expression

As previously described, the production of therapeutically effective rAAV particles is not a completely efficient process. AAV production results in a mixture of therapeutically effective rAAV particles, therapeutically ineffective rAAV particles, and production impurities (e.g., low molecular weight DNA and small nucleotides, extrinsic high molecular weight DNA, buffer components, etc.). Therapeutically effective rAAV particles are capable of infecting cells such that the infected cells express (e.g., by transcription and/or by translation) an element (e.g. nucleotide sequence, protein, etc.) of interest. To this extent, the therapeutically effective rAAV particles can include AAV particles having capsids or vgs with different properties. For example, the therapeutically effective rAAV particles can have capsids with different post translation modifications. In other examples, the therapeutically effective rAAV particles can have vgs with differing sizes/lengths, plus or minus strand sequences, different flip/flop inverted terminal repeat (ITR) configurations, different number of ITRs, or truncations. Therapeutically effective rAAV particles are also referred to as “heavy”, “full”, or “partial” capsids. Therapeutically ineffective rAAV particles are incapable of infecting cells or a cell infected with therapeutically ineffective rAAV particles are unable to express (e.g., by transcription and/or by translation) an element (e.g. nucleotide sequence, protein, etc.) of interest. Therapeutically ineffective rAAV particles can contribute to decreased effectiveness per unit dose of capsid and can increase the risk of an immune response due to a needed increased number of foreign proteins being introduced into the patient for an effective amount of heavy/full capsid. Therapeutically ineffective rAAV particles can include AAV particles having capsids or vgs with different properties and are referred to as empty capsids or light capsids. For example, empty capsids do not have a vg or have an unquantifiable vg concentration. Empty or light capsids can also have different capsid properties. While not being bound to by any particular theory, the heavy/full/partially full capsids differ from light or empty capsids in their charge and/or density.

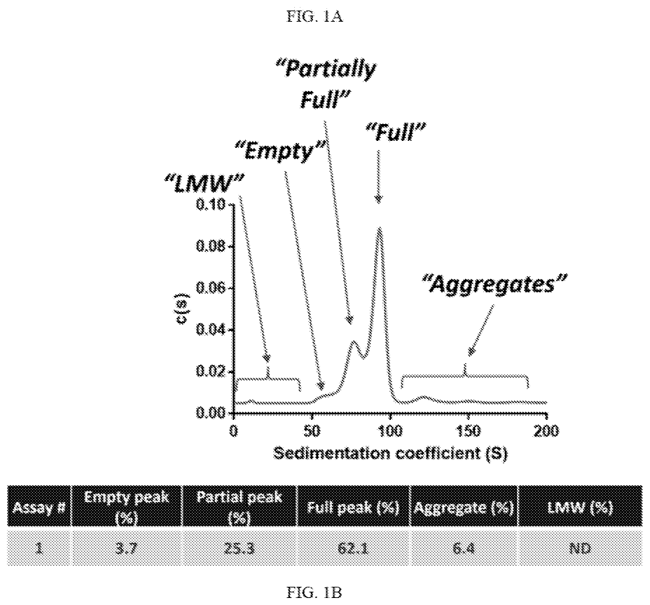

FIGS. 1A and 1B show an example particle profile of an AAV preparation using analytical ultracentrifugation. As shown in FIG. 1B, it can be seen that some low molecular weight impurities may be present, but the main impurities are about 4% empty capsids and about 6-7% aggregates. Therapeutically effective capsids from the preparation include about 62% full/heavy capsids and about 25% partially full/partial capsids.

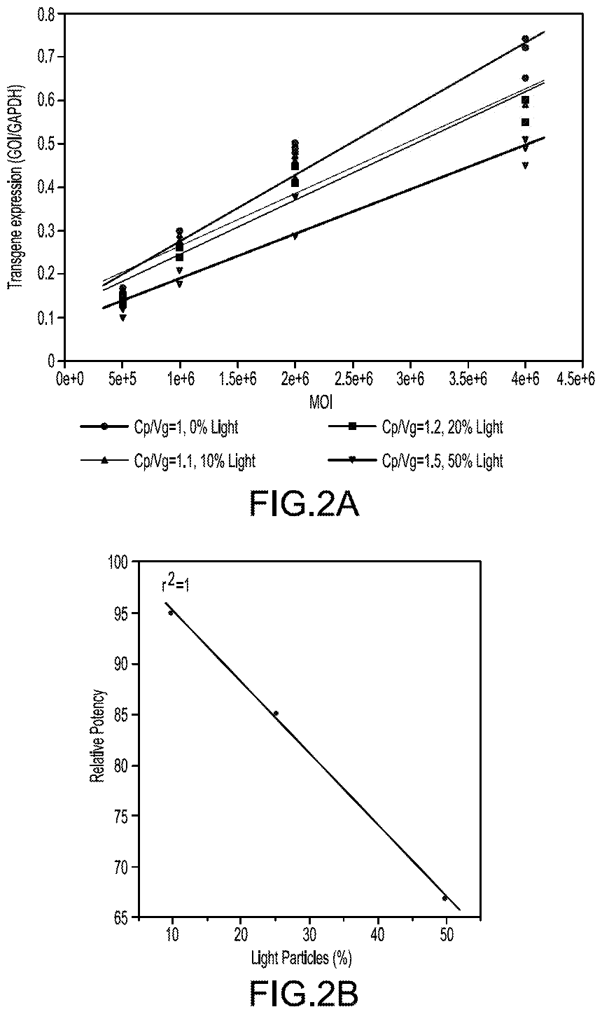

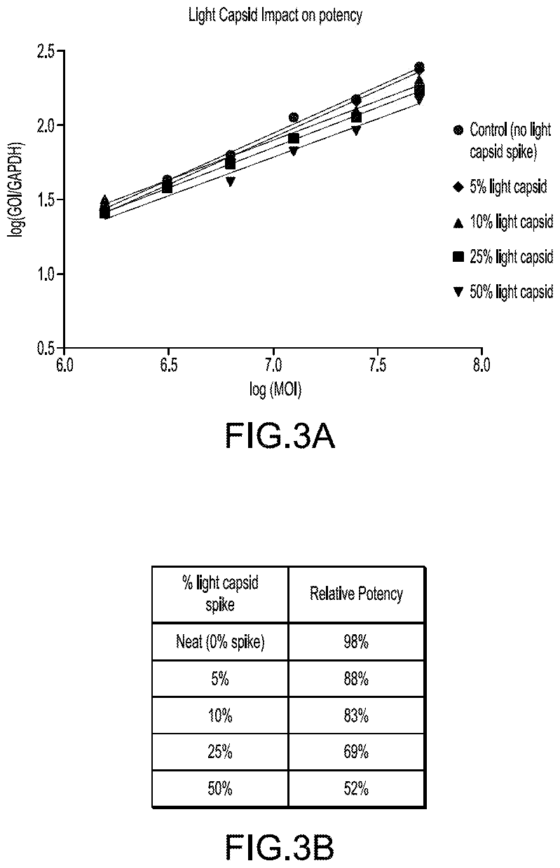

FIGS. 2A, 2B, 3A, and 3B show the effect of the presence of light particles on transgene expression of a gene of interest. HepG2 cells were infected with therapeutically effective rAAV particles (i.e., heavy capsids) with a transgene for gene of interest #1 (GOI1) and known concentrations of therapeutically ineffective rAAV particles (i.e., light capsids). As shown in FIGS. 2A, 2B, 3A, and 3B, increasing concentrations of the light capsids reduced transgene expression in HepG2 cells. FIG. 3B highlights that the relative potency is reduced by about 48% when the light capsids make up about 50% of the capsids infecting cells. Thus, the presence of light particles reduces transgene expression and potency of heavy particles in HepG2 cells.

FIGS. 4, 5A, 5B, 6A, 6B, 7, 8A, 8B, 9A, 9B, 10, 11A, 11B, 11C, 11D, 12A, and 12B show that light capsids can bind HepG2 cells, can enter the HepG2 cell, and can enter the nucleus of the HepG2 cell. For this study, heavy capsids were labelled with a Cyanine 3 (Cy3) dye that exhibits green fluorescence or Cyanine 5 (Cy5) dye that exhibits red fluorescence. Light capsids were also labelled with Cy3 or Cy5. Specifically, the labeling reacting Cy3/Cy5 NHS esters with primary amines on the heavy/light capsids to yield stable bonds.

The labeled particles were added to HepG2 cells. Specifically, HepG2 cells were seeded on a cell imaging plate prior to AAV transduction. The cells were incubated with labeled AAVs for 1 hour at 4° C. to bind the cell surface receptor and prevent internalization by inhibiting endocytosis. The cells were incubated at 37° C. for 4-8 hours to allow heavy and light capsids to transduce the HepG2 cells. The cells were then washed with CMEM and PBS, fixed with 4% PFA for 10 minutes, washed three more time with PBS, mounted with an anti-fading agent for confocal microscopy.

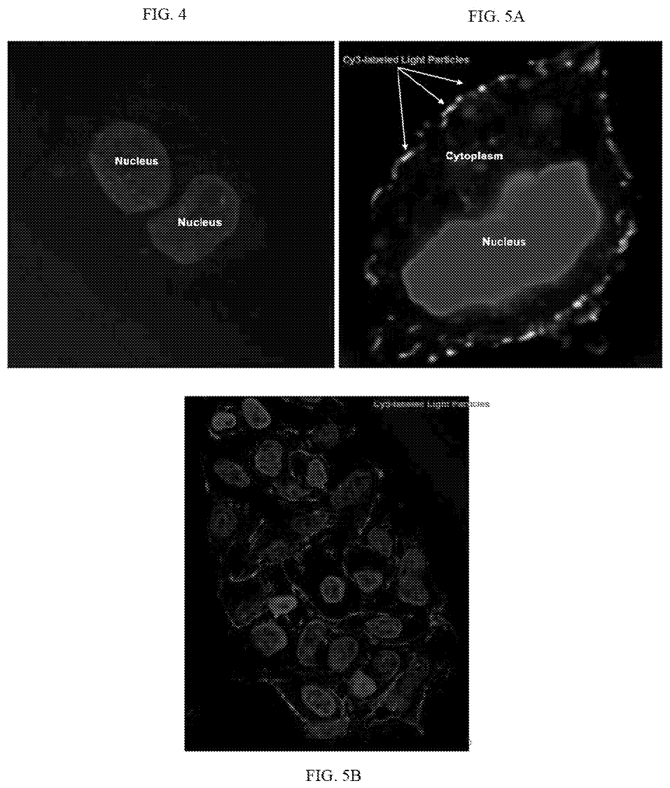

FIG. 4 shows a control without Cyanine dye not showing a strong signal, whereas FIGS. 5A and 5B show Cy3-labelled and Cy5-labelled light particles bound to HepG2 cell surface receptors, respectively.

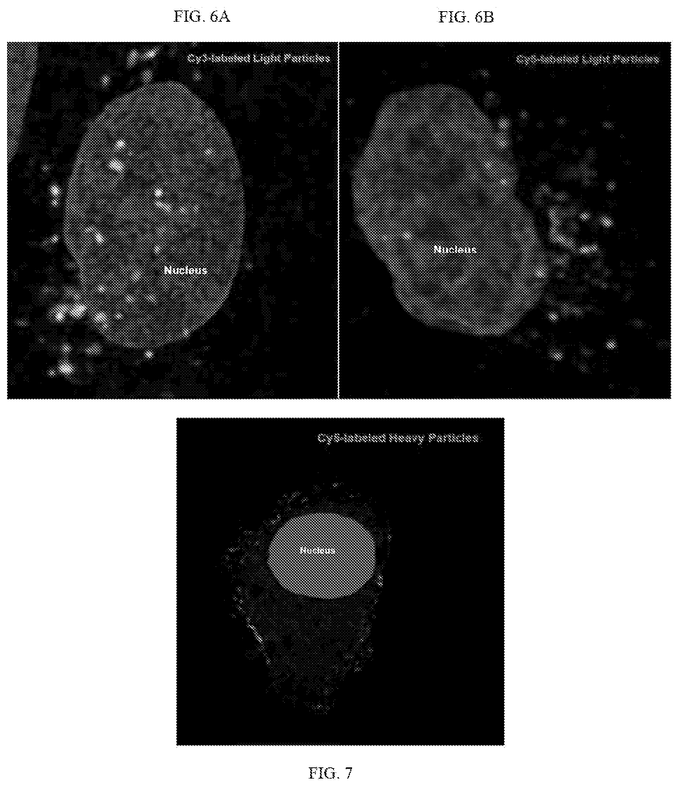

FIGS. 6A and 6B show Cy3-labelled (left panel) and Cy5-labelled (right panel) light particles are detected in the nucleus of the HepG2 cell after 8 hours incubation at 37° C.

FIG. 7 shows Cy5-labelled heavy particles bound to HepG2 cell surface receptors.

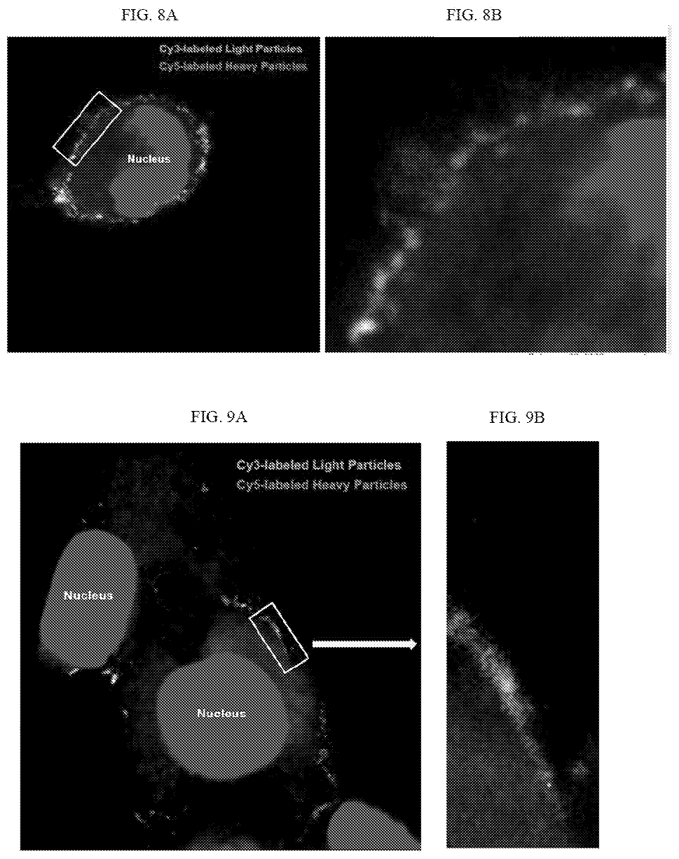

FIGS. 8A, 8B, 9A, and B show Cy3-labelled light particles and Cy5-labelled heavy particles both bind HepG2 cell surface receptors.

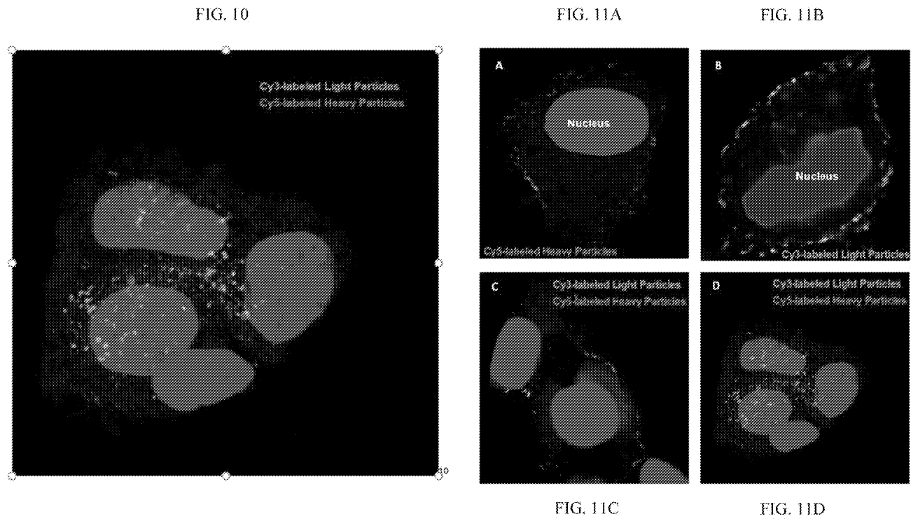

FIG. 10 shows Cy3-labelled light particles and Cy5-labelled heavy particles are detected in the nucleus of the HepG2 cell after 8 hours of incubation at 37° C.

FIGS. 11A, 11B, 11C, and 11D shows Cy3-labelled light particles and Cy5-labelled heavy particles both bind HepG2 cell surface receptors after 1 hour at 4° C. FIGS. 11A, 11B, and 11C are at 1 hour at 4° C., with red being Cy5-labelled heavy particles and green being C3-labeled light particles. FIG. 11D shows heavy and light particle binding in the nucleus of HepG2 cells after 8 hours at 37° C.

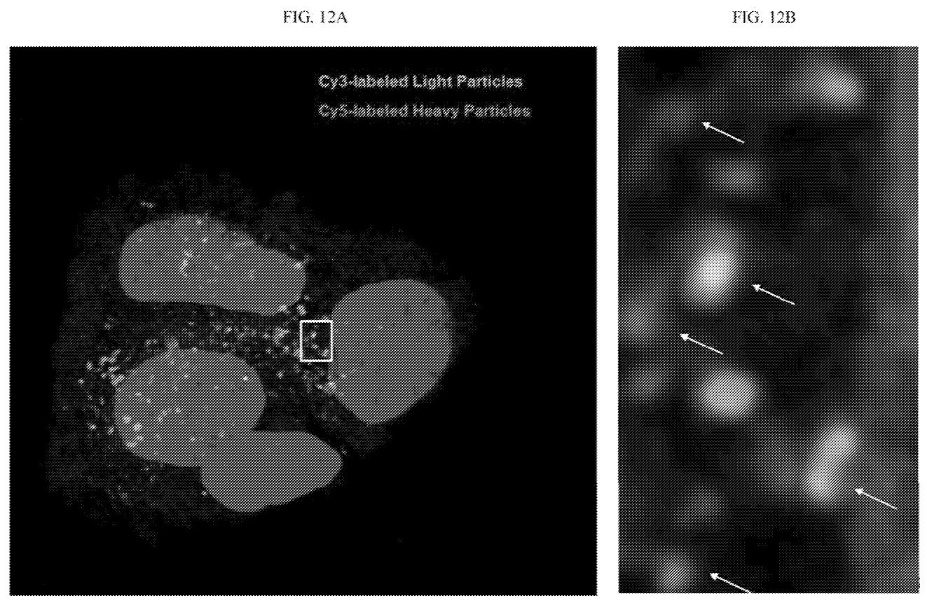

As noted above, FIG. 11D shows heavy and light particle binding in the nucleus of HepG2 cells after 8 hours at 37° C. FIG. 11D also shows that Cy3-labelled light particles and Cy5-labelled heavy particles are detected in the nucleus of the HepG2 cell after 8 hours incubation at 37° C. As shown in FIGS. 12A and 12B, the highlighted portions (see box and arrows) show co-localization of Cy3 and Cy5 dyes, which may indicate that the heavy and light particles are using the same cell machinery. This co-localization may explain the reduced efficacy caused by the presence of light capsid and further demonstrates the desire to obtain an AAV preparation that is free or substantially free, i.e., greater than 99%, preferably greater than 99.5% free of light capsid.

In view of the negative effects of light and empty capsids, their removal is important for increasing the efficacy of AAV gene therapeutics.

### Example 2

### Purification of AAV Capsids Encoding of Gene of Interest #1, Gene of Interest #2, and Gene of Interest #3

The following example discloses the production of rAAV with either GOI1, gene of interest #2 (GOI2), or gene of interest #3 (GOI3). GOI1 has a polynucleotide size of less than 5.5 Kb. GOI2 is a polynucleotide size of less than 6 Kb. GOI3 has a polynucleotide size of less than 5.5 Kb and is different from GOI1. The rAAV with GOI1 and GOI2 were pseudotyped with AAVS capsids. The rAAV with G013 were pseudotyped with AAV9 capsids.

All downstream column and TFF operations are performed at ambient temperature. Intermediate pools are held at cooled temperature if extended hold times are necessary.

Cell-free culture fluid (“harvest pool material”) containing AAV capsids with GOI1, GOI2, or GO3 was used as a starting material.

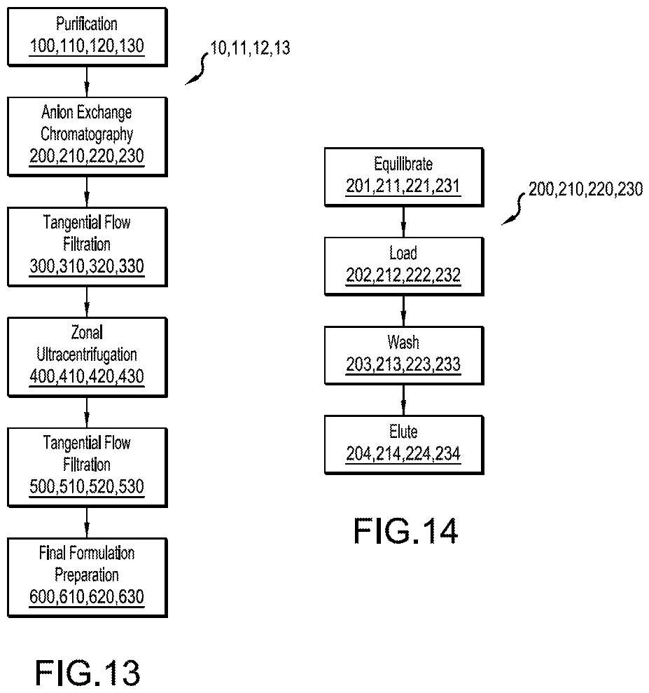

As shown in FIG. 13, the processing 10 of harvest pool material includes purifying 100 capsids from the harvest pool material and processing capsids via AEX 200, TFF 300, ZUC 400, TFF 500, and preparing the final formulation of the capsids 600. The processing 11 of harvest pool material containing rAAV with GOI1 pseudotyped with AAVS capsids includes purifying 110 capsids from the harvest pool material and processing capsids via AEX 210, TFF 310, ZUC 410, TFF 510, and preparing the final formulation of the capsids 610. The processing 12 of harvest pool material containing rAAV with G012 pseudotyped with AAVS capsids includes purifying 120 capsids from the harvest pool material and processing capsids via AEX 220, TFF 320, ZUC 420, TFF 520, and preparing the final formulation of the capsids 620. The processing 13 of harvest pool material containing rAAV with GO13 pseudotyped with AAV9 capsids includes purifying 130 capsids from the harvest pool material and processing capsids via AEX 230, TFF 330, ZUC 430, TFF 530, and preparing the final formulation of the capsids 620.

In step 100,101,102,103 of FIG. 13, rAAV is purified from the harvest pool material for subsequent AEX and ZUC processing. For example, purifications steps 110,120 include processing harvest pool material for GOI1 and GO2 using AVB immunochromatography for affinity purification of AAVS capsids. In another example, purifications step 130 includes processing harvest pool material for GOI3 using column immunochromatography for affinity purification of AAV9 capsids. Affinity column purified AAV capsids is used for AEX and ZUC processing.

As shown in FIG. 14 and in step 200 of FIG. 13, the isolated capsids having a mixture of heavy, partial, light, and empty AAV capsids is subjected to Anion Exchange Chromatography (AEX) using AEX column a polymeric, strong or weak anion exchange column (AEX column). The AEX step 200 includes the steps of equilibrating the column 201, loading the column with the harvest pool material 202, washing the column to remove impurities 203, and eluting and isolating AAV capsids from the AEX column 204. In an example for GOI1, the AEX step 210 includes the steps of equilibrating the column 211, loading the column with the harvest pool material 212, washing the column to remove impurities 213, and eluting and isolating AAV capsids from the AEX column 214. In an example for GOI2, the AEX step 220 includes the steps of equilibrating the column 221, loading the column with the harvest pool material 222, washing the column to remove impurities 223, and eluting and isolating AAV capsids from the AEX column 224. In an example for GOI3, the AEX step 230 includes the steps of equilibrating the column 231, loading the column with the harvest pool material 232, washing the column to remove impurities 233, and eluting and isolating AAV capsids from the AEX column 234.

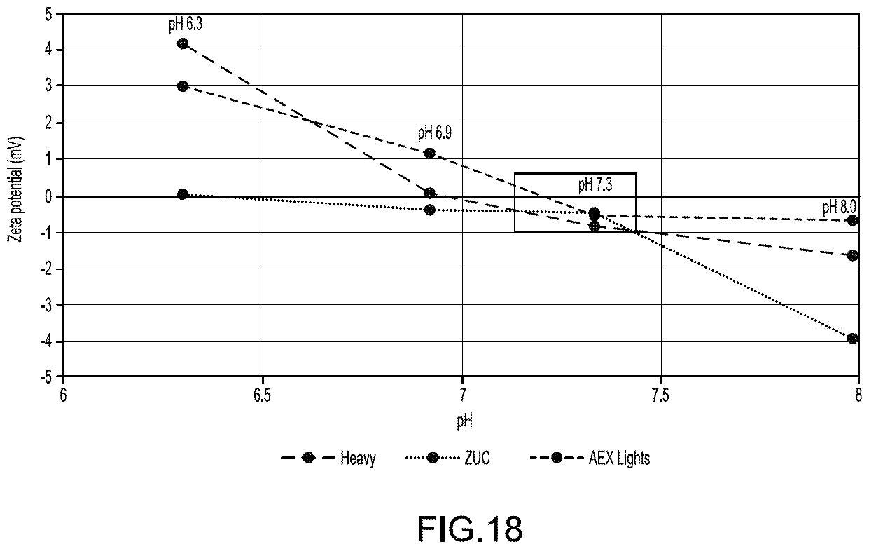

The zeta potential for the following capsids at different pH is analyzed: heavy capsids for GOI1, GOI2, and GOI3; light capsids extracted after AEX elution 204,214,224,234; and ZUC processed capsids from step 400,410,420,430. As shown in FIG. 18 for GOI2, it was discovered that there is a light capsid population that can be removed using AEX since the net negative charge of these light capsids is lower than the heavy capsids. This light capsid population also could not be detected by general 260 nm/280 nm absorbance measurements and requires more precise measurements (i.e. size exclusion chromatography) for detection.

Buffers and solutions used for AEX separation are known in the art. Examples of such buffers include: an AEX equilibration buffer having a conductivity of ≤1 mS/cm or 1-7 mS/cm and a pH ranging from 7-10, an AEX wash buffer having a conductivity ranging from 4-7 mS/cm and a pH ranging from 7-9, an AEX elution buffer having a conductivity ranging from 5-10 mS/cm and a pH ranging from 7-9, an AEX strip buffer having a conductivity ranging from 53.2-70.1 mS/cm, and an AEX elution pool adjustment buffer having a pH ranging from 6-9.

The following parameters is used for the AEX column: a load capacity ranging from 0.1×10e16 to 10×10e16 vg/L; a column with a strong anionic exchange resin and a bed height ranging from 7-15 cm; and a flow rate ranging from 50-160 cm/hr.

The isolated capsids are filtered with 0.22 micron (μm) filters prior to and after AEX processing.

Prior to the AEX separation step, the pH of the isolated capsids is adjusted with an Adjustment buffer to a pH ranging from 7 to 9 and a conductivity of ≤3 mS/cm.

The AEX column is prepared with a volume of AEX Equilibration Buffer. The post-column pH and conductivity is checked for a pH ranging from 7-10 and a conductivity <1 mS/cm or 1-7 mS/cm.

The load pool is applied and the column is washed with AEX Equilibration Buffer and AEX Wash Buffer. The column is manually observed to identify when the absorbances of the eluted AEX Wash Buffer reached A₂₆₀=A₂₈₀. If the A₂₆₀=A₂₈₀ crossover is not observed, additional AEX Wash Buffer is added to the AEX column.

The AEX elution is accomplished by adding a volume of the AEX Elution buffer at the AEX column.

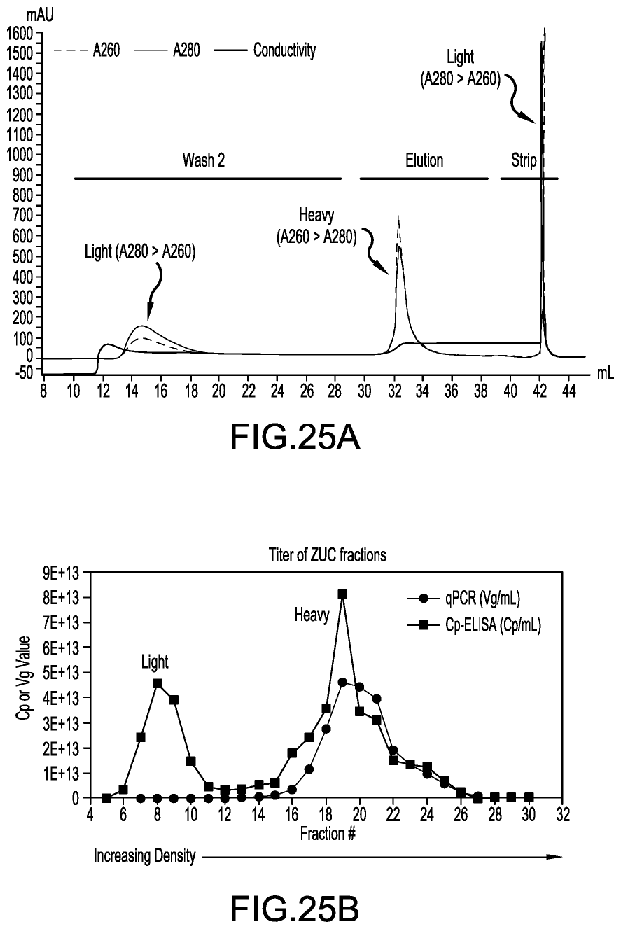

The A₂₆₀ and A₂₈₀ profile of the AEX processing 200,210,220,230 for GOI1, GOI2, and GOI3 is visualized and analyzed. FIG. 25A shows A₂₆₀ and A₂₈₀ profile of the AEX processing for the rAAV preparation for GOI2 during the wash, elution, and strip steps.

The pH of the Elution Pool was adjusted to 6-9 with a volume of the AEX elution pool adjustment buffer.

Generally, vg and capsid (cp) titers may be evaluated in any way that is suitable for measuring the respective vg and capsids. For example, quantitative polymerase chain reaction (qPCR) may be used to measure vg titers and enzyme-linked immunosorbent assay (ELISA) may be used to measure Cp titer. Alternatively, SEC (size-exclusion chromatography)-HPLC may be used to measure the vg and cp titers. In addition, RP (reverse phase)-HPLC assay may be used to evaluate the potential impact of process parameters on VP ratios.

qPCR may be used for vg quantification by quantitative polymerase chain reaction (qPCR) using a standard qPCR system, such as an Applied Biosystems 7500 Fast Real-Time PCR system. Alternatively, digital droplet PCR (ddPCR) may be used for Vg quantification. Primers and probes may be designed to target the DNA of the AAV, allowing its quantification as it accumulates during PCR. Examples of ddPCR are described in Pasi, K. John, et al. “Multiyear Follow-Up of AAV5-hFVIII-SQ Gene Therapy for Hemophilia A.” New England Journal of Medicine 382.1 (2020): 29-40 Regan, John F., et al. “A Rapid Molecular Approach for Chrorriosornal Phasing.” PloS one 10.3 (2015). e0118270; and Furuta-Hanav,ia, Birei, Temhide Yamaguchi, and Eriko Uchida. “Two-Dimensional Droplet Digital PCR as a Tool for Titration and Integrity Evaluation of Recombinant Adeno-Associated Viral Vectors” Human gene therapy methods 30.4 (2019): 127-136. Other systems for vg quantification include SEC, SEC-HPLC, and size exchange chromatography multi-angle light scattering, all of which are described in WO 2021/062164, which is incorporated in its entirety by reference.

The capsid ELISA (cp-ELISA) assay measures intact capsids using, e.g., the AAVS Capsid ELISA method and may utilize a commercially-available kit (for example, Progen PRAAVS). This kit ELISA employs a monoclonal antibody specific for a conformational epitope on assembled AAVS or other capsids. Capsids can be captured on a plate-bound monoclonal antibody, followed by subsequent binding of a detection antibody. The assay signal may be generated by addition of conjugated streptavidin peroxidase followed by addition of colorimetric TMB substrate solution, and sulfuric acid to end the reaction. The titers of test samples are interpolated from a four-parameter calibration curve of the target capsid standard. Another system for quantifying capsid titers is SEC-MALS, which are described in WO 2021/062164.

Heavy and partial AAV capsids may be measured using techniques known in the art. For example, the total number of capsids may be measured using cp-ELISA using antibodies specific to capsid proteins. Heavy and partial capsids may be measured using qPCR to measure the vector genome present.

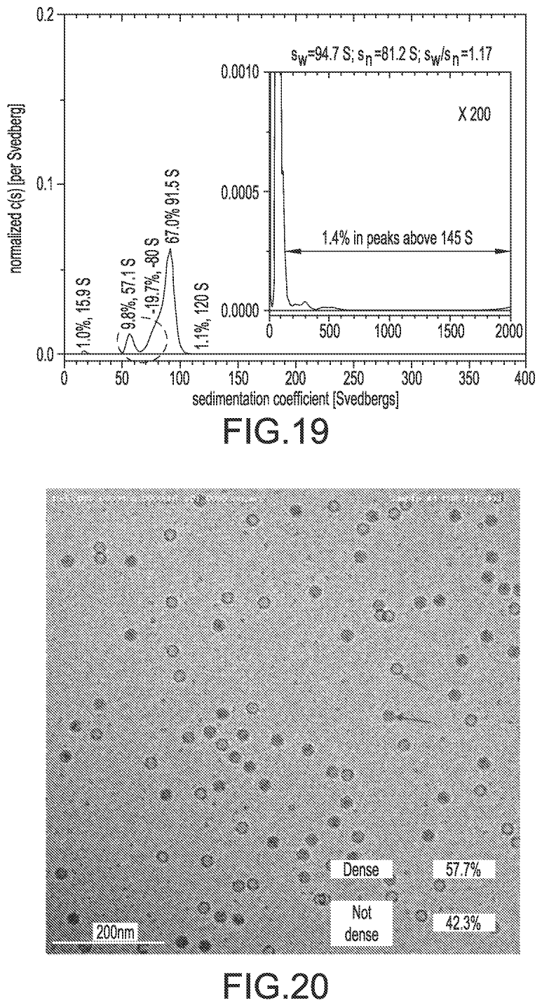

The particle distribution profile from preparations of GOI1, GOI2, and GOI3 after anion exchange chromatography 200,210,220,230 is analyzed. FIG. 19 shows processing the rAAV preparation of GOI2 with AEX reduced the light and empty capsids concentration to 9.8%. Cryogenic electron microscopy images from preparations of GOI1, GOI2, and GOI3 after anion exchange chromatography 200,210,220,230 is also analyzed. As shown in FIG. 20, the capsid count from cryogenic electron microscopy images of the rAAV preparation for GOI2 post AEX processing was 57.7% dense particles (i.e., heavy and partial capsids) and 42.3% “not dense” particles (i.e., light capsids). Accordingly, AEX does not remove all of the light capsids from an rAAV preparation.

The removal of contaminating virus by AEX processing 200,210,220,230 is also assessed by adding known concentrations of contaminating virus to the isolated capsids and processing the composition through the AEX column. As shown in Table 1, AEX processing of the rAAV preparation of GOI1 reduced the concentration of contaminating virus by a Logio reduction of at least 2.

TABLE 1
Log10 Reduction of Contaminating Virus after AEX Processing

| Virus | Log₁₀ Reduction |
|---|---|
| VSV | >5.5 |
| X-MuLV | >5.7 |
| AcNPV | >4.7 |
| SV-40 | >4.7 |
| Reo-3 | >5.8 |
| PPV | >5.9 |
| EMC | >2.0 |

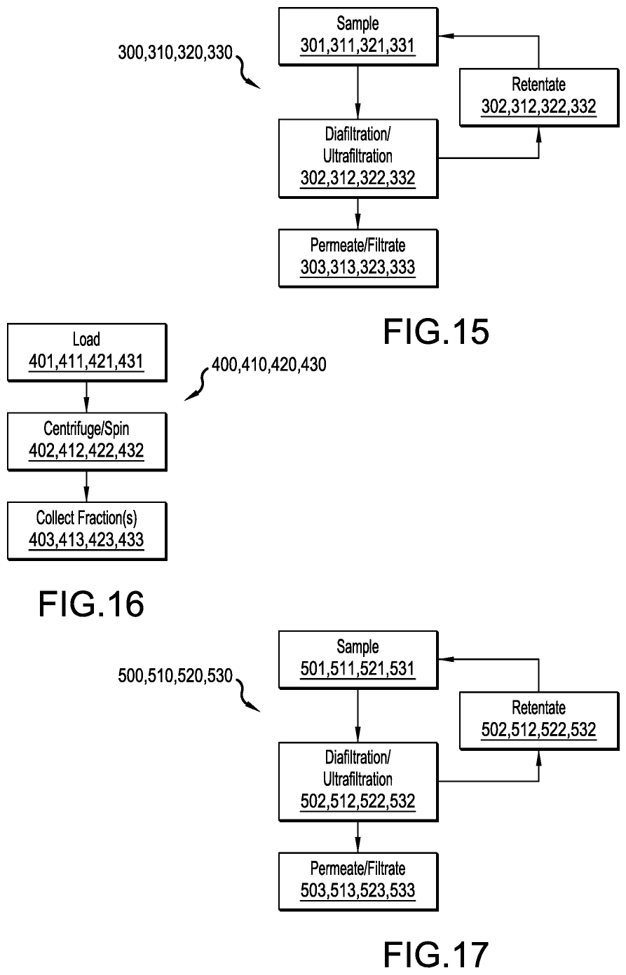

Following AEX, the collected elution pool is subjected to a first tangential-flow filtration (TFF) Ultrafiltration/Diafiltration (UF/DF) in step 300 of FIG. 13. The AEX column elution pool was concentrated and diafiltered into TFF buffer in preparation for zonal ultracentrifugation. As shown in FIG. 15, TFF UF/DF 300 includes the steps of providing a sample 301 (e.g., the eluate 204) and diafiltering/ultrafiltering 302 the sample into a permeate/filtrate 303 or a retentate 302 that can be returned to the samples 301. For GOI1, TFF UF/DF 310 includes the steps of providing a sample 311 (e.g., the eluate 214) and diafiltering/ultrafiltering 312 the sample into a permeate/filtrate 313 or a retentate 312 that can be returned to the samples 311. For GOI2, TFF UF/DF 320 includes the steps of providing a sample 321 (e.g., the eluate 224) and diafiltering/ultrafiltering 322 the sample into a permeate/filtrate 323 or a retentate 322 that can be returned to the samples 321. For GOI3, TFF UF/DF 330 includes the steps of providing a sample 331 (e.g., the eluate 234) and diafiltering/ultrafiltering 332 the sample into a permeate/filtrate 333 or a retentate 332 that can be returned to the samples 331.

A load ranging from 0.1×10e17 vg/m² to 10×10e17 vg/m² is loaded onto a ultrafiltered and diafiltered with a 100 kD molecular weight cut off (MWCO) membrane, where process was controlled by TMP and crossflow.

The diafiltered pool is subject to 0.2 μm filtration with using a 0.22 uM PVDF filter generating a final TFF Pool.

The TFF Pool may be optionally frozen at ≤60° C. before the ZUC processing if a long hold time is desired.

Following TFF UF/DF 300 and as shown in FIG. 16, the TFF pool is processed by ZUC 400 including the steps of loading the zonal rotor 401 with the composition 302 and component used for forming a gradient (e.g., cesium chloride, etc.), centrifuging the loaded rotor 402, and collected the identified fractions 403. For GOI1, ZUC 410 including the steps of loading the zonal rotor 411 with the composition 312 and component used for forming a gradient (e.g., cesium chloride, etc.), centrifuging the loaded rotor 412, and collected the identified fractions 413. For GOI2, ZUC 420 including the steps of loading the zonal rotor 421 with the composition 322 and component used for forming a gradient (e.g., cesium chloride, etc.), centrifuging the loaded rotor 422, and collecting the identified fractions 423. For GOI3, ZUC 430 including the steps of loading the zonal rotor 431 with the composition 332 and component used for forming a gradient (e.g., cesium chloride, etc.), centrifuging the loaded rotor 432, and collected the identified fractions 433.

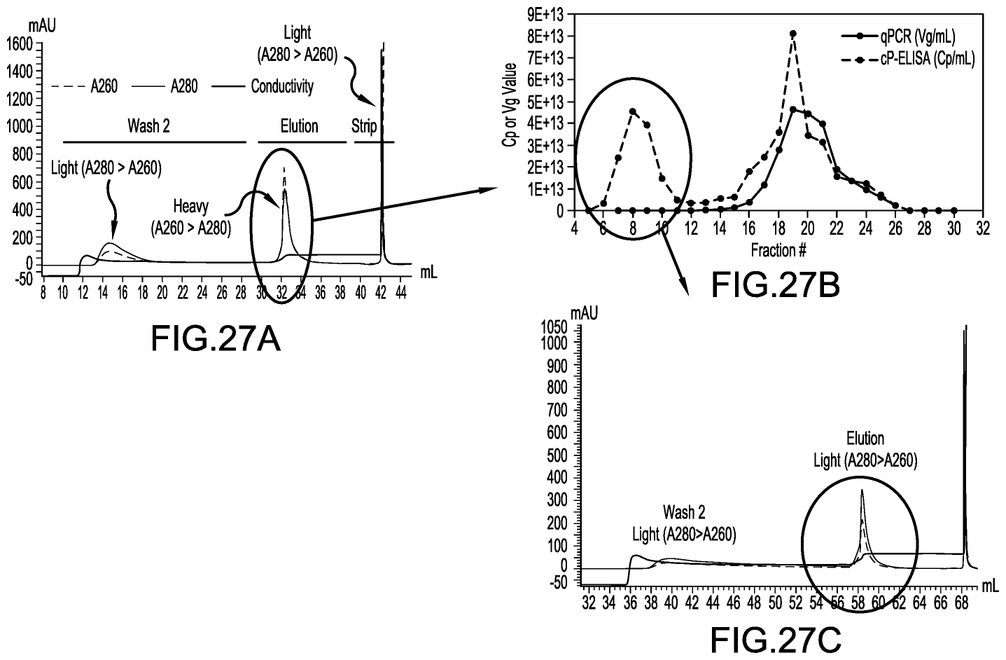

rAAV (i.e., GOI1, GOI2, and GOI3) preparations are processed by AEX 200,210,220,230 and ZUC 400,410,420,430. Fractions containing ZUC light capsids are collected and re-processed by AEX, where the A₂₆₀ and A₂₈₀ profile was visualized. As shown in FIGS. 27A, 27B, and 27C for the rAAV preparation of GOI2, it was also discovered that a population of light capsids elutes with heavy capsids during AEX. This population of light capsids can be further separated from the full capsids using zonal ultracentrifugation (ZUC) since qPCR shows that these capsids do not have any quantifiable encapsulated DNA even though the net negative charge of these capsids is the same as the heavy capsid. In step 400 of FIG. 13, the TFF product was subject to ZUC by diluting the TFF product pool with TFF-A buffer and adjusting with a CsCl buffer to a final concentration of ranging from 15% to 75% CsCl. An overlay solution with a CsCl concentration (15% to 75% CsCl) that is less than the cesium chloride of the product pool adjusted with CsCl was pumped into the centrifuge rotor, followed by the product pool adjusted with CsCl, then by a cushion solution with a CsCl concentration (15% to 75% CsCl) that is less than the cesium chloride of the product pool adjusted with CsCl. A CsCl gradient was formed during the spin, partitioning product forms with different densities. Fractions from the gradient were recovered from the rotor. ZUC was performed at ambient temperature. Product pool was collected automatically based on in-line density measurement, or by analysis of recovered fractions. The ZUC was performed using the following parameters.

For GOI1, ZUC rotors are loaded with compositions having titers ranging from 0.1×10e17 vg/load to 10×10e17 vg/load. The ZUC parameters include centrifuging the composition at a speed ranging for 80000 G to 125000 G for 14-16 hrs at a temperature ranging from 10° C. to 36° C.

For GOI2, ZUC rotors are loaded with compositions having titers ranging from 0.1×10e16 vg/load to 10×10e16 vg/load or 0.5×10e16 vg/load to 50×10e16 vg/load. The ZUC parameters include centrifuging the composition at a speed ranging from 10000 rpm to 50000 rpms for 15-25 hrs at a temperature ranging from 10° C. to 36° C. Alternatively, one of the ZUC parameters includes centrifuging the composition at a speed ranging from 50000 G to 100000 G.

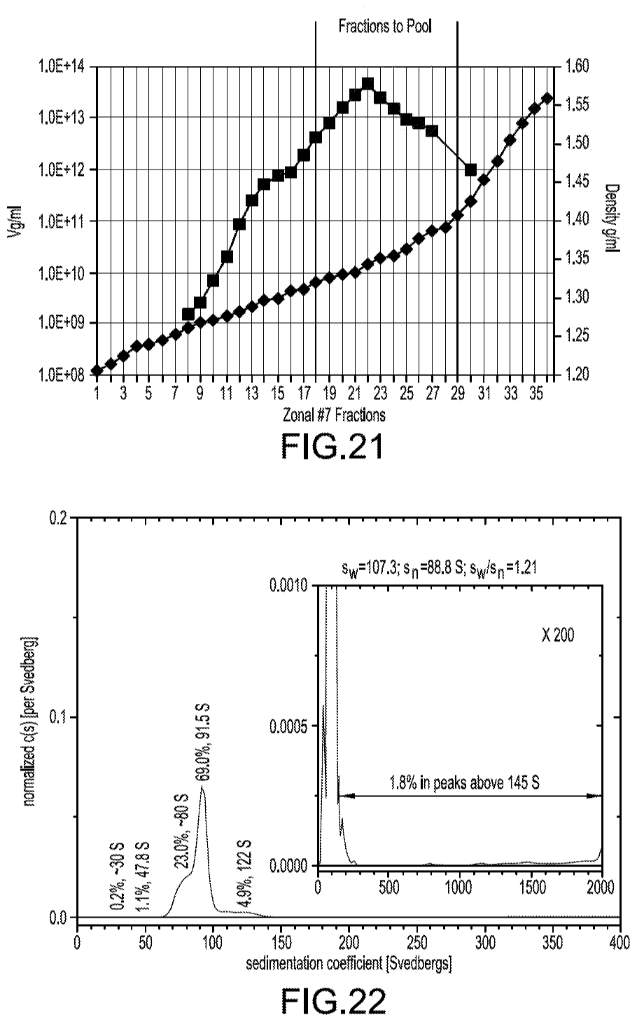

Vg concentration relative to densities of the different collected fractions 403,413,423,433 of rAAV (i.e., GOI1, GOI2, and GOI3) is processed via ZUC 400,410,420,430 were determined and analyzed. FIG. 21 shows increasing concentrations of vgs up to fraction 22, where fractions 18-29 were identified to have increased concentrations of heavy and partial capsids for the rAAV preparation of GOI2. It was noted that fractions 18-29 had densities ranging from greater than 1.30 g/mL to ≤1.45 g/mL. The particle distribution profile from the different collected fractions 403,413,423,433 of rAAV (i.e., GOI1, GOI2, and GOI3) is processed via ZUC 400,410,420,430 were also analyzed. FIG. 22 shows processing the rAAV preparation with ZUC reduced the light capsids concentration to 1.1%.

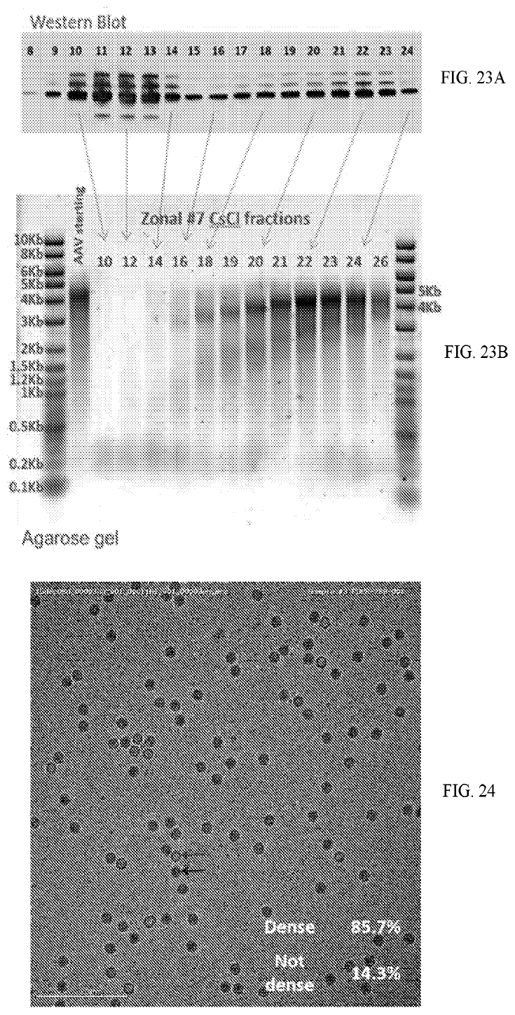

The rAAV (i.e., GOI1, GOI2, and GOI3) after ZUC 400,410,420,430 is analyzed by Western blots, alkaline agarose gels, and cryogenic electron microscopy. FIG. 23A and 23B show high density fractions 18-24 have significantly greater concentrations of capsids with larger genome sizes (i.e., heavy and partial capsids) than lower density fractions 10-16 for the rAAV preparation of GOI2. As shown in FIG. 24, the capsid count from cryogenic electron microscopy images of the rAAV preparation for GOI2 post ZUC processing was 85.7% dense particles (i.e. heavy and partial capsids) and 14.3% “not dense” particles (i.e. light capsids). Accordingly, ZUC alone does not remove all of the light capsids from an rAAV preparation.

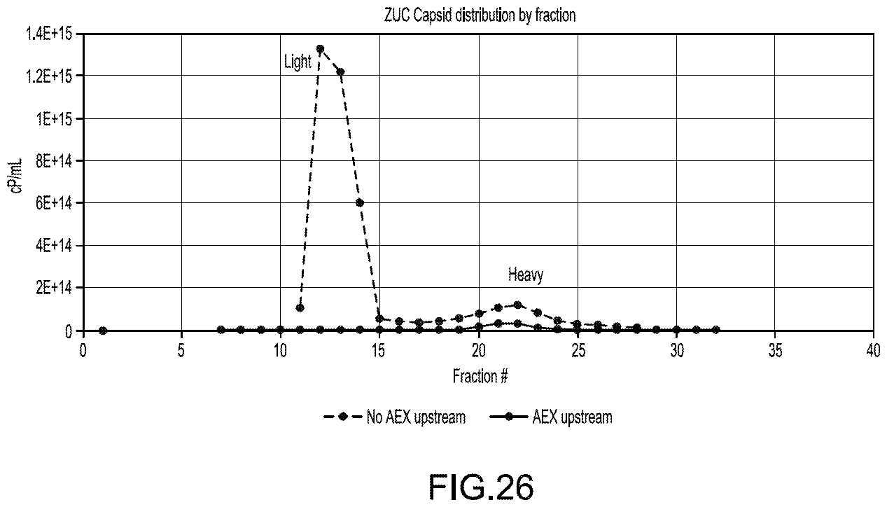

Vg concentration of the different collected fractions 403,413,423,433 of rAAV (i.e., GOI1, GOI2, and GOI3) is processed via ZUC 400,410,420,430 were determined by qPCR, ddPCR, SEC, SEC-HPLC or SEC-MALS. Capsid titers of the different collected fractions 403,413,423,433 of rAAV (i.e., GOI1, GOI2, and GOI3) is processed via ZUC 400,410,420,430 were determined by cp-ELISA or SEC-MALS. For rAAV preparation for GOI2, FIG. 25B shows the capsid titer of each fraction as determined by cp-ELISA and vg titer of each fraction as determined by qPCR. As shown in FIG. 25B, fractions 15-26 has greater capsids and vg titers as compared to the other fractions. Capsid titers of rAAV (i.e., GOI1, GOI2, and GOI3) processed with or without AEX 200,210,220,230 were determined by cp-ELISA. FIG. 26 also shows that AEX essentially reduces light and empty capsids such that the ZUC processing is not overloaded with the light and empty capsids.

Table 2 shows the properties of the rAAV preparation of GOI2 after ZUC processing with different titer loadings.

TABLE 2

| | | ZUC Load Range | | |
|---|---|---|---|---|
| | | | ZUC load of | ZUC load of |
| Assays | | TFF-A | 0.5E16 vg | 3.0E16 vg |
| SEC-HPLC | % HMW DNA | 0.8 | 0.4 | 0.2 |
| | % Dimer | 4.4 | 1.2 | 1.1 |
| | % Monomer | 94.8 | 98.4 | 98.7 |
| RP-HPLC | % VP2 | 8.4 | 8.2 | 8.1 |
| | % VP3 | 87.5 | 87.7 | 87.9 |
| | % VP1 | 4.0 | 4.0 | 4.0 |
| AUC | % Small | 0 | 0 | 0 |
| | % Light | 19.5 | 0 | 0 |
| | % Intermediates | 15.8 | 24.5 | 19.9 |
| | % Heavy | 55.5 | 72.1 | 68.7 |
| | % Aggregates | 9.2 | 3.4 | 11.4 |

Table 3 shows the properties of another rAAV preparation of GOI2 after ZUC processing with different titer loadings.

TABLE 3

| | Actual Vg | Pool Density | % Vg | Cp/Vg | % |
|---|---|---|---|---|---|
| ZUC Run | Loaded [Vg] | [g/mL] | Recovery | Ratio | Dimer |
| ZUC Run A | 4.2E15 | 1.4078 | 71 | 1.0 | 1.5 |
| 0.5E16 vg | | | | | |
| ZUC Run E | 2.8E16 | 1.4090 | 80 | 1.0 | 1.5 |
| 3.0E16 vg | | | | | |

Analysis of the reduction of contaminating virus by ZUC processing is also assessed for GOI1, GOI2, and GOI3. For baculovirus, ZUC processing reduced by a Logio concentration of ≥2 (e.g., 2.09 and 2.46).

In step 500 of FIG. 13, the ZUC elution pool is concentrated and diafiltered into a stabilizing TFF-B buffer. As shown in FIG. 17, TFF UF/DF 500 includes the steps of providing a sample 501 (e.g., the collected fractions 403) and diafiltering/ultrafiltering 502 the sample into a permeate/filtrate 503 or a retentate 502 that can be returned to the samples 501. For GOI1, TFF UF/DF 310 includes the steps of providing a sample 511 (e.g., the collected fractions 413) and diafiltering/ultrafiltering 512 the sample into a permeate/filtrate 513 or a retentate 512 that can be returned to the samples 511. For GOI2, TFF UF/DF 520 includes the steps of providing a sample 521 (e.g., the collected fractions 423) and diafiltering/ultrafiltering 522 the sample into a permeate/filtrate 523 or a retentate 522 that can be returned to the samples 521. For GOI3, TFF UF/DF 530 includes the steps of providing a sample 531 (e.g., the collected fractions 433) and diafiltering/ultrafiltering 532 the sample into a permeate/filtrate 533 or a retentate 532 that can be returned to the samples 531. A load ranging from 0.1×10e17 vg/m² to 10×10e17 vg/m² is loaded onto an ultrafiltered and diafiltered with a 100 kD molecular weight cut off (MWCO) membrane, where the process was controlled by TMP and crossflow.

In step 600 of FIG. 13, the combined TFF product pool material is diluted into a formulation buffer to a predetermined concentration and filtered through a 0.2 μm filter.

The combined AEX (FIG. 25A) and ZUC (FIG. 25B) processing of the rAAV preparation of GOI2 obtains an essentially pure preparation of heavy and partial capsids of about 1.0 Cp/Vg ratio.

For the rAAV preparation of GOI1, an analytical ultracentrifugation analysis is conducted on the rAAV production processed by AEX and ZUC. The capsid composition in the processed rAAV production was 0% light capsids, 3.9% capsid aggregates, 7.68% intermediate or partially full capsids, and 88.4% heavy capsids.

### Example 3

### Removal of AAV Production Impurities: rAAV Associated with Rep Protein(s) and Deamidated Capsids

It was discovered that Rep protein, particularly Rep78 and Rep68, remains associated to a concentration of rAAV after production. This rAAV associated with Rep78/Rep68 is an impurity that may be incapable of infecting cells or a cell infected with the rAAV associated with Rep78/Rep68 may be unable to express (e.g., by transcription and/or by translation) an element (e.g., nucleotide sequence, protein, etc.) of interest. The rAAV associated with Rep78/Rep68 may contribute to decreased effectiveness per unit dose of capsid and may increase the risk of an immune response due to a needed increase of foreign proteins being introduced into the patient for an effective amount of heavy/full/partially full capsid. Accordingly, AEX and ZUC processing reduces the concentration of rAAV associated with Rep78/Rep68. The rAAV associated with Rep78/Rep68 of from GOI1, GOI2, and GOI3 preparations is quantified by Liquid Chromatography-Mass Spectrometry (LC-MS). The assay accurately measures Rep78/Rep68 concentrations. Capsids are isolated after AVB processing, AEX processing, and ZUC processing. The capsids are denatured to dissociate viral proteins and digested to peptides prior to LC-MS analysis. Peptides from Rep78/Rep68 proteins are separated on LC and resulting signal from multiple fragments of the targeted peptides are analyzed by a triple quadrupole mass spectrometer.

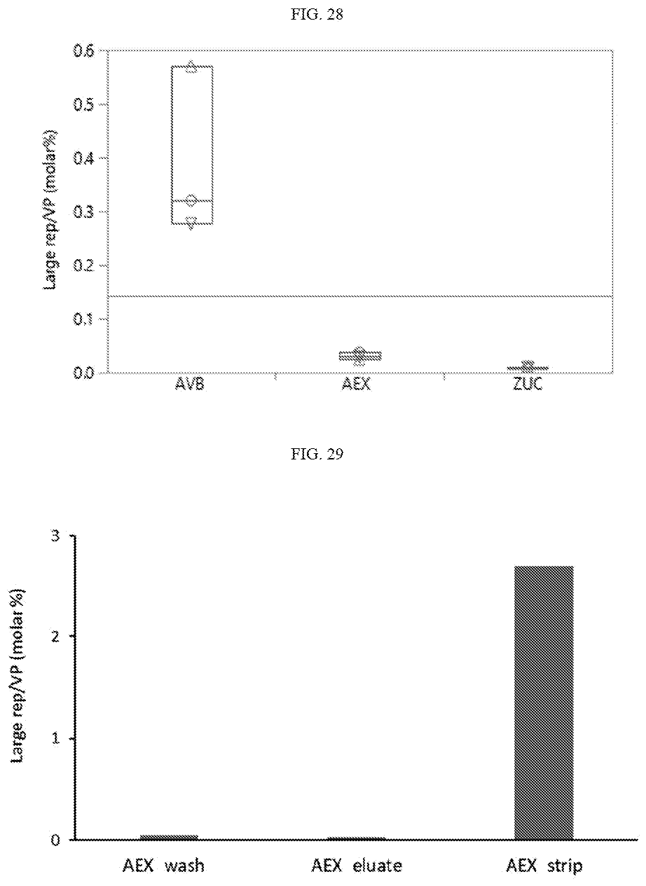

As shown in FIG. 28, a concentration of rAAV associated with Rep protein(s) elutes with therapeutically effective rAAV during AVB immunochromatography for AAV5 capsids. For GOI1, AEX processing substantially reduced the concentration of rAAV associated with Rep protein(s), but some of the rAAV associated with Rep protein(s) eluted with the therapeutically effective rAAV. After AEX, ZUC processing further reduced the concentration of rAAV associated with Rep protein(s). For example, AEX and ZUC processing removed the concentration of rAAV associated with Rep protein(s) such that the final composition containing therapeutically effective rAAV is substantially devoid of rAAV associated with Rep protein(s).

As shown in FIG. 29 for GOI1, rAAV associated with Rep protein(s) remains within the AEX column after the washing and elution steps. The rAAV associated with Rep protein(s) exits the AEX column only when the column is regenerated.

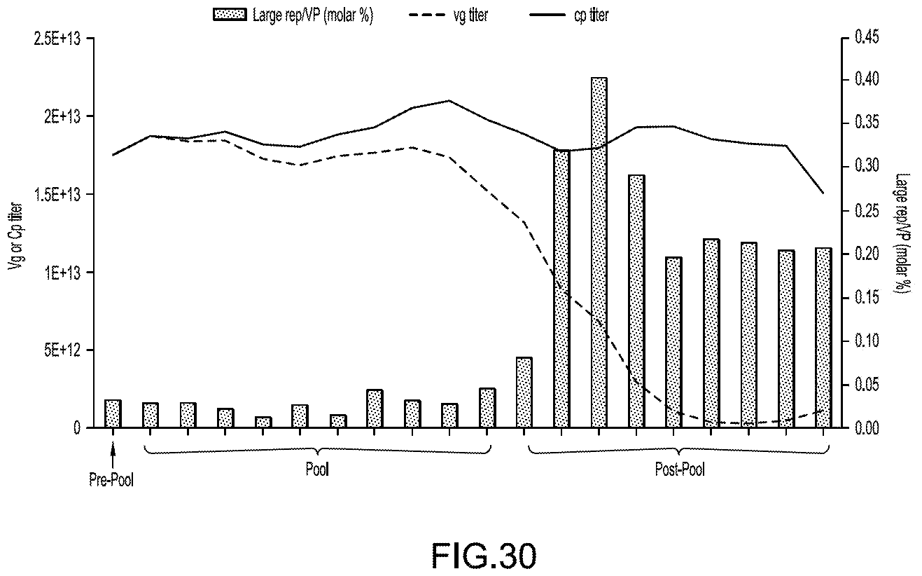

As shown in FIG. 30 for GOI1, ZUC processing also separates therapeutically effective rAAV from rAAV associated with Rep protein(s) such that the therapeutically effective rAAV can be purified from rAAV associated with Rep protein(s). Particularly, “Pool” fractions that are isolated contain little to no rAAV associated with Rep protein(s), whereas “Post-pool” fractions contain substantially greater concentrations of rAAV associated with Rep protein(s). It is also noted that the rAAV associated with Rep protein(s) are empty or light capsids.

It has been shown that deamidation of capsids reduces rAAV infectivity of the expression the transgene provided by the rAAV (Giles, April R., et al. Molecular Therapy 26.12 (2018): 2848-2862) and Frederick, Amy, et al. Human Gene Therapy 31.13-14 (2020): 756-774. Thus, rAAV with deamidated capsids (or deamidated rAAV) are impurities that may be incapable of infecting cells or a cell infected with the deamidated rAAV may be unable to express (e.g. by transcription and/or by translation) an element (e.g. nucleotide sequence, protein, etc.) of interest. Deamidated rAAV may also contribute to decreased effectiveness per unit dose of capsid and may increase the risk of an immune response due to a needed increase of foreign proteins being introduced into the patient for an effective amount of heavy/full/partially full capsid. Accordingly, AEX and ZUC processing reduces the concentration of rAAV with deamidated capsids. The deamidation level of VP1 protein at the N-terminus of GOI1, GOI2, and GOI3 are quantified by Liquid Chromatography-Mass Spectrometry (LC-MS). The assay accurately measures percent deamidation at the N-terminal region of VP1. Capsids are isolated after AVB processing, AEX processing, and ZUC processing. The capsids are denatured to dissociate viral proteins and digested to peptides prior to LC-MS analysis. Unmodified and deamidated forms of the target VP1 N-terminal peptide bearing the target deamidation sites are separated on LC and resulting signal from multiple fragments of the targeted peptide are analyzed by a triple quadrupole mass spectrometer.

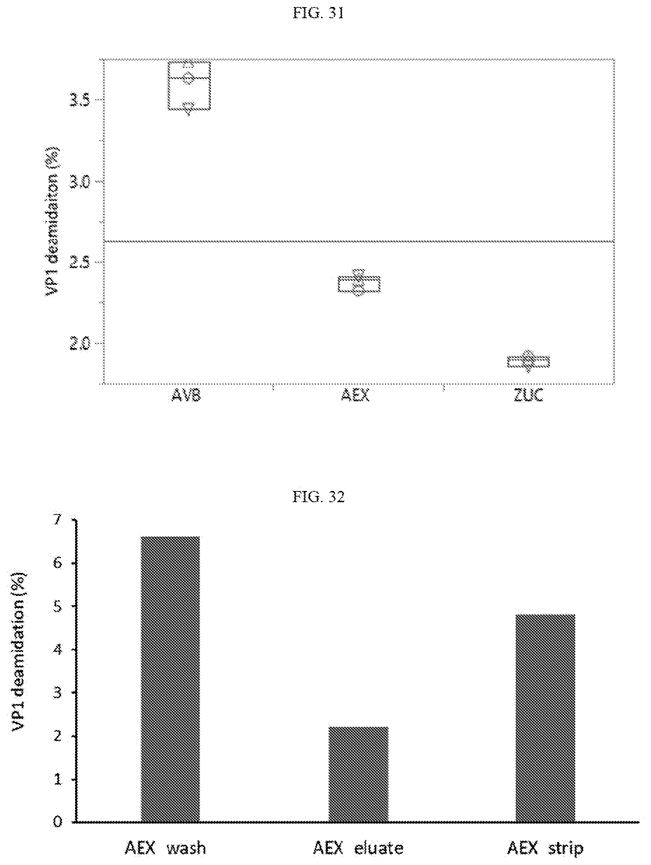

As shown in FIG. 31 for GOI1, a concentration of deamidated rAAV elutes with therapeutically effective rAAV during AVB immunochromatography for AAV5 capsids. AEX processing substantially reduced the concentration of deamidated rAAV, but some of the deamidated rAAV eluted with the therapeutically effective rAAV. After AEX, ZUC processing further reduced the concentration of deamidated rAAV. For example, AEX and ZUC processing removed the concentration of deamidated rAAV such that the final composition containing therapeutically effective rAAV is substantially devoid of deamidated rAAV.

As shown in FIG. 32 for GOI1, deamidated rAAV is removed during the washing step and exits the AEX column only when the column is regenerated. It is also noted that the AEX eluate has a substantially reduced concentration of deamidated rAAV.

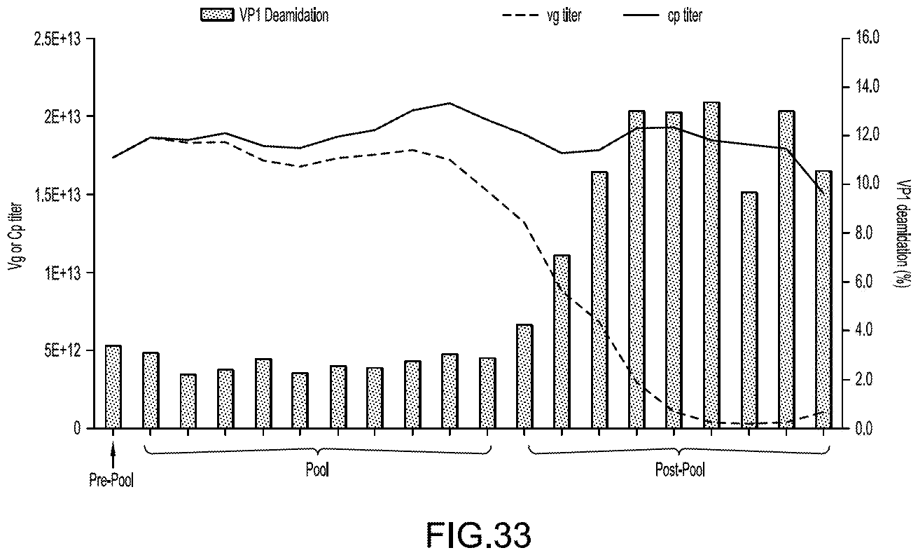

As shown in FIG. 33 for GOI1, ZUC processing also separates therapeutically effective rAAV from deamidated rAAV such that the therapeutically effective rAAV can purified be deamidated rAAV. Particularly, “Pool” fractions that are isolated contain reduced concentrations of deamidated rAAV, whereas “Post-pool” fractions contain substantially greater concentrations of deamidated rAAV. It is also noted that the deamidated rAAV are empty or light capsids.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A method of purifying therapeutically effective recombinant adeno-associated virus (rAAV) particles, the method comprising the steps of:
providing a composition including therapeutically effective rAAV particles and AAV production impurities, where a first portion of the AAV production impurities comprises impurities having a net charge different from the AAV particles and a second portion of the AAV production impurities comprises impurities having a density different from the AAV particles;
removing the first portion from the composition by anion-exchange chromatography; and
removing the second portion from the composition by zonal ultracentrifugation;
wherein, after anion-exchange chromatography and zonal ultracentrifugation, the composition is substantially devoid of AAV production impurities.

2. The method of claim 1, wherein the composition after anion-exchange chromatography and zonal ultracentrifugation is at least 95% pure from AAV production impurities.

3. The method of claim 1, wherein the composition after anion-exchange chromatography and zonal ultracentrifugation is at least 99% pure from AAV production impurities.

4. The method of claim 1, wherein the composition after anion-exchange chromatography and zonal ultracentrifugation is 99+% pure from AAV production impurities.

5. The method of claim 1, wherein the removing the first portion from the composition by anion-exchange chromatography allows for the removal of the second portion from the composition by zonal ultracentrifugation.

6-16. (canceled)

17. The method of claim 1, wherein the anion-exchange chromatography is a polystyrene/divinyl benzene resin.

18. The method of claim 17, wherein the resin is modified with quaternary ammonium groups.

19. The method of claim 1 wherein the zonal ultracentrifugation uses a cesium chloride gradient.

20. The method of claim 19, wherein the zonal ultracentrifugation with a cesium chloride gradient comprises
adding a concentration of cesium chloride to the elute;
overlaying a first cesium chloride solution in a spinning centrifuge rotor, the first cesium chloride solution having a cesium chloride concentration that is less than the cesium chloride concentration of the elute;
adding elute from the anion-exchange ion chromatography;
adding a second cesium chloride solution, the second cesium chloride solution having a cesium chloride concentration that is greater than the cesium chloride concentration of the elute;
centrifuging the spinning centrifuge rotor to form a density gradient within the elute; and
collecting fractions from the density gradient.

21. A method of purifying therapeutically effective recombinant adeno-associated virus (rAAV) particles, the method comprising the steps of:
providing a composition including therapeutically effective rAAV particles and therapeutically ineffective rAAV particles;
removing at least some of the therapeutically ineffective rAAV particles from the composition by anion-exchange chromatography; and
processing the composition by zonal ultracentrifugation;
wherein, after anion-exchange chromatography and zonal ultracentrifugation, the composition is substantially devoid of therapeutically ineffective rAAV particles.

22. The method of claim 21, wherein the removal step allows for subsequent processing of the composition by zonal ultracentrifugation.

23. The method of claim 21, wherein the removal step allows the composition of the providing step to have a greater quantity of therapeutically effective rAAV particles that are processed by zonal ultracentrifugation.

24. (canceled)

25. The method of claim 21, wherein the removal step removes at least 50% of the therapeutically ineffective rAAV particles from the composition.

26. The method of claim 21, wherein the composition after anion-exchange chromatography and zonal ultracentrifugation is at least 95% pure from therapeutically ineffective rAAV particles.

27. The method of claim 21, wherein the composition after anion-exchange chromatography and zonal ultracentrifugation is free from any detectable therapeutically ineffective rAAV particles.

28. The method as in claim 21, wherein the therapeutically ineffective rAAV particles comprise capsids associated with Rep proteins.

29-31. (canceled)

32. The method as in claim 21, wherein the therapeutically ineffective rAAV particles comprise capsids with VP1, VP2, or VP3 capsid proteins having a deamidated amino acid.

33. The method as in claim 21, wherein the therapeutically ineffective rAAV particles comprise capsids devoid of a vector genome or encapsulating an undetectable concentration of nucleotide.

34. The method as in claim 21, wherein the therapeutically ineffective rAAV particles comprise capsids with vector genomes having one or more sizes that are insufficient for cells infected by the capsids to generate therapeutically effective nucleotide sequences.

35. The method as in claim 21, wherein the therapeutically ineffective rAAV particles comprise capsids with vector genomes having one or more sizes that reduce expression of an element by a cell infected with the capsids and therapeutically effective rAAV encoding the element relative to expression of the element by a cell infected under the same conditions but being devoid of the infection with the capsid.
